(19) United States
(12) Patent Application Publication
(10) Pub. No.: US 2016/0174533 A1
(43) Pub. Date: Jun. 23, 2016

(54) GENETICALLY MODIFIED RAT MODELS FOR DRUG METABOLISM

(71) Applicant: Transposagen Biopharmaceuticals, Inc., Lexington, KY (US)

(72) Inventors: Eric M. Ostertag, Del Mar, CA (US); John Stuart Crawford, Lexington, KY (US)

(21) Appl. No.: 15/040,254

(22) Filed: Feb. 10, 2016

Related U.S. Application Data
(63) Continuation of application No. 12/850,921, filed on Aug. 5, 2010.
(60) Provisional application No. 61/231,549, filed on Aug. 5, 2009.

(51) Int. Cl. A01K 67/027

(52) U.S. Cl. CPC A01K 67/0276; A01K 2207/05; A01K 2217/075; A01K 2217/15; A01K 2227/105; A01K 2267/03

(57) ABSTRACT

The present invention provides a desired rat or a rat cell which contains a predefined, specific and desired alteration rendering the rat or rat cell predisposed to alterations in drug and chemical metabolism by modification of its structure or mechanism. Specifically, the invention pertains to a genetically altered rat, or a rat cell in culture, that is defective in at least one of two alleles of a drug metabolism gene such as the Cyp7b1 gene, the Cyp3a4 gene, etc. In another embodiment, the rat cell is a somatic cell. The inactivation of at least one drug metabolism allele results in an animal with a higher susceptibility to altered drug and chemical metabolism. In one embodiment, the genetically altered animal is a rat of this type and is able to serve as a useful model for altered drug and chemical metabolism or toxicology and as a test animal for autoimmune and other studies. The invention additionally pertains to the use of such rats or rat cells, and their progeny in research and medicine. In one embodiment, the invention provides a genetically modified or chimeric rat cell whose genome comprises two chromosomal alleles of a drug metabolism gene wherein at least one of the two alleles contains a mutation, or the progeny of the cell.

### CROSS-REFERENCE TO RELATED APPLICATIONS

This application claims the benefit of U.S. Provisional Patent Application Ser. No. 61/231,549, filed Aug. 5, 2009, which application is hereby incorporated by reference in its entirety for all purposes.

### BACKGROUND OF THE INVENTION

Gene modification is a process whereby a specific gene, or a fragment of that gene, is altered. This alteration of the targeted gene may result in a change in the level of RNA and/or protein that is encoded by that gene, or the alteration may result in the targeted gene encoding a different RNA or protein than the untargeted gene. The modified gene may be studied in the context of a cell, or, more preferably, in the context of a genetically modified animal.

Genetically modified animals are among the most useful research tools in the biological sciences. An example of a genetically modified animal is a transgenic animal, which has a heterologous (i.e., foreign) gene, or gene fragment, incorporated into their genome that is passed on to their offspring. Although there are several methods of producing genetically modified animals, the most widely used is microinjection of DNA into single cell embryos. These embryos are then transferred into pseudopregnant recipient foster mothers. The offspring are then screened for the presence of the new gene, or gene fragment. Potential applications for genetically modified animals include discovering the genetic basis of human and animal diseases, generating disease resistance in humans and animals, gene therapy, toxicology studies, drug testing, pharmacokinetics and production of improved agricultural livestock.

Identification of novel genes and characterization of their function using mutagenesis has also been shown to be productive in identifying new drugs and drug targets. Creating in vitro cellular models that exhibit phenotypes that are clinically relevant provides a valuable substrate for drug target identification and screening for compounds that modulate not only the phenotype but also the target(s) that controls the phenotype. Modulation of such a target can provide information that validates the target as important for therapeutic intervention in a clinical disorder when such modulation of the target serves to modulate a clinically relevant phenotype.

The major organs for human drug metabolism and elimination are the liver and intestine which contain the preponderance of metabolic enzymes, including the cytochome P450 (CYP) family of enzymes. CYP enzymes are largely responsible for the metabolism and biotransformation of drugs in humans and rats. CYPs are often the limiting factor in the therapeutic relevance of a drug. In some cases the biotransformation of drugs by CYP enzymes results in the generation of toxic metabolites. Similarly, CYP enzymes can create structural changes to produce more active compounds from the parent compound, for example, by O-methylation. CYP's play a vital role in manipulating drug-drug interactions or drug-substance interactions on a competitive and mechanism basis. Therefore, the effect of drug metabolism by CYP enzymes can dictate the drug's efficacy and safety. These enzymes are also involved in a number of physiological functions such as, steroid, bile acid, vitamin, prostaglandins, and xenobiotic metabolism. CYP's can also dictate the localization and accumulation of a particular substance. CYP's are important for the metabolism of exogenous chemicals, including carcinogens. CYP's often display altered expression and activity in different environments; such as disease states and diet fluctuations. Genetically modified animal models of human drug metabolism can be used to predict the clearance and toxicity of drugs and other substances in humans. One requirement for the discovery and development a therapeutic agent is the establishment of the average pharmacokinetic and dynamic response to a particular drug. Once the response is determined a dose range can then be calculated to determine the range of therapeutic effectiveness versus toxicity. The tighter the margin of safety, as for many oncoceuticals or cancer drugs, the more accurate the dose response must be. Since cancer drugs currently hold a failure rate of around 97% the development of accurate drug metabolism animal models is crucial. Humans, rats, and other model organisms have a significant number of orthologous CYP genes which exhibit similar or identical functions. Many xenobiotic metabolizing CYP genes display interspecies conservation with respect to substrate specificity and gene regulation. Therefore, animal models which embody a deficiency or genetic modification in CYP genes can used to study biotransformation, pharmacology, toxicology, and carcinogenesis of a particular drug or chemical.

Another important application of genetically modified CYP's in rats is the ability to determine human risk to chemicals. These studies elucidate what chemicals and derivatives may be toxic or carcinogenic in humans. Many CYP enzymes are involved in the metabolism of toxic compounds such as carcinogens. Genetically modified CYP rats can be used to discover the mechanism of bioactivation. Studies can be done on different CYP deficient rats to delineate which enzyme is responsible for increased or decreased carcinogenesis. In several CYP knockout models, protection from toxicity is displayed. These models are compared to wild-type to discover the mechanism of bioactivation and inactivation of compounds. The in vivo rat models are very important in studies of chemical risk. The complex nature of chemical and xenobiotic metabolism is extremely difficult to reproduce in an in vitro model.

Animal models of genetically modified CYP metabolism genes are also useful to evaluate drug and chemical metabolism differences associated with diet and disease states. Several CYP expression levels are increased during starvation and in diabetes patients. These CYP enzymes produce metabolites which are a part of the gluconeogenic pathway. Several CYP inhibitors are known to reduce gluconeogenesis; however, inhibitor specificity was not determined. When CYP knockouts were studied in model organisms, only one model, Cyp2e1−/− mice, exhibited reduced gluconeogenesis. The model was instrumental in the discovery of CYP metabolism alteration during fasting and diabetes. This same method can be used to determine the correlation of genetic differences in populations with the effect on metabolism of drugs and other compounds. Populations which have a prevalent single nucleotide polymorphism (SNP) can be susceptible to altered drug and compound metabolism. Animal models which reflect these populations by containing the same polymorphisms can be used to predict the differences in compound metabolism among populations.

Another benefit of the CYP genetically modified rat models is the ability to study organ specific sequestration of chemicals and drugs. In humans and animal studies the environmental contaminant TCDD, which is an aromatic hydrocarbon has been shown to accumulate in the liver. When the Cyp1a2 knockout mouse was exposed to TCDD it displayed very little hepatic accumulation and severally increased adipose accumulation when compared to wild-type. The CYP knockout model determined the mechanism of TCDD organ sequestration. Sequestration and elimination is an important aspect of drug and chemical safety and efficacy. Toxicology genes such as CYP's can be studied to determine if, for example, a known neurotoxin derivative will accumulate in the brain or be eliminated. These models are essential for determine the mechanism of toxicity for environmental chemicals and drugs.

Animal models exhibiting clinically relevant phenotypes are also valuable for drug discovery and development and for drug target identification. For example, mutation of somatic or germ cells facilitates the production of genetically modified offspring or cloned animals having a phenotype of interest. Such animals have a number of uses, for example as models of physiological disorders (e.g., of human genetic diseases) that are useful for screening the efficacy of candidate therapeutic compounds or compositions for treating or preventing such physiological disorders. Furthermore, identifying the gene(s) responsible for the phenotype provides potential drug targets for modulating the phenotype and, when the phenotype is clinically relevant, for therapeutic intervention. In addition, the manipulation of the genetic makeup of organisms and the identification of new genes have important uses in agriculture, for example in the development of new strains of animals and plants having higher nutritional value or increased resistance to environmental stresses (such as heat, drought, or pests) relative to their wild-type or non-mutant counterparts.

Since most eukaryotic cells are diploid, two copies of most genes are present in each cell. As a consequence, mutating both alleles to create a homozygous mutant animal is often required to produce a desired phenotype, since mutating one copy of a gene may not produce a sufficient change in the level of gene expression or activity of the gene product from that in the non-mutated or wild-type cell or multicellular organism, and since the remaining wild-type copy would still be expressed to produce functional gene product at sufficient levels. Thus, to create a desired change in the level of gene expression and/or function in a cell or multicellular organism, at least two mutations, one in each copy of the gene, are often required in the same cell.

In other instances, mutation in multiple different genes may be required to produce a desired phenotype. In some instances, a mutation in both copies of a single gene will not be sufficient to create the desired physiological effects on the cell or multi-cellular organism. However, a mutation in a second gene, even in only one copy of that second gene, can reduce gene expression levels of the second gene to produce a cumulative phenotypic effect in combination with the first mutation, especially if the second gene is in the same general biological pathway as the first gene. This effect can alter the function of a cell or multi-cellular organism. A hypomorphic mutation in either gene alone could result in protein levels that are severely reduced but with no overt effect on physiology. Severe reductions in the level of expression of both genes, however, can have a major impact. This principle can be extended to other instances where mutations in multiple (two, three, four, or more, for example) genes are required cumulatively to produce an effect on activity of a gene product or on another phenotype in a cell or multi-cellular organism. It should be noted that, in this instance, such genes may all be expressed in the same cell type and therefore, all of the required mutations occur in the same cell. However, the genes may normally be expressed in different cell types (for example, secreting the different gene products from the different cells). In this case, the gene products are expressed in different cells but still have a biochemical relationship such that one or more mutations in each gene is required to produce the desired phenotype.

### BRIEF SUMMARY OF THE INVENTION

In accordance with the purposes of this invention, as embodied and broadly described herein, this invention relates to the engineering of animal cells, preferably mammalian, more preferably rat, that are deficient due to the disruption of gene(s) or gene product(s) resulting in altered drug and chemical metabolism or toxicology.

In another aspect, the invention relates to genetically modified rats, as well as the descendants and ancestors of such animals, which are animal models of human drug metabolism, efficacy, toxicity and methods of their use.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWING

This invention, as defined in the claims, can be better understood with reference to the following drawings:

FIGS. 1-4 show the process for creating a genetically modified drug metabolism rat model using DNA transposons to create an insertion mutation directly in the germ line.

FIG. 1: Gene modification by DNA transposons.

FIG. 2: Breeding strategy for creating rat knockouts directly in the germ cells with DNA transposons.

FIG. 3: DNA sequences

FIG. 4: DNA transposon-mediated insertion mutation in Rattus norvegicus Cyp7b1 gene.

In the following description of the illustrated embodiments, references are made to the accompanying drawings, which form a part hereof, and in which is shown by way of illustration various embodiments in which the invention may be practiced. It is to be understood that other embodiments may be utilized and structural and functional changes may be made without departing from the scope of the present invention.

### DETAILED DESCRIPTION OF THE INVENTION

Definitions

The present invention may be understood more readily by reference to the following detailed description of preferred embodiments of the invention and the Examples included therein and to the Figures and their previous and following description. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention. Nothing herein is to be construed as an admission that the invention is not entitled to antedate such disclosure by virtue of prior invention.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Throughout this application, reference is made to various proteins and nucleic acids. It is understood that any names used for proteins or nucleic acids are art-recognized names, such that the reference to the name constitutes a disclosure of the molecule itself.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

“Complementary,” as used herein, refers to the subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

A “deletion mutation” means a type of mutation that involves the loss of genetic material, which may be from a single base to an entire piece of chromosome. Deletion of one or more nucleotides in the DNA could alter the reading frame of the gene; hence, it could result in a synthesis of a nonfunctional protein due to the incorrect sequence of amino acids during translation.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed”. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene”, also called a “structural gene” means a DNA sequence that codes for or corresponds to a particular sequence of amino acids which comprise all or part of one or more proteins or enzymes, and may or may not include introns and regulatory DNA sequences, such as promoter sequences, 5′-untranslated region, or 3′-untranslated region which affect for example the conditions under which the gene is expressed. Some genes, which are not structural genes, may be transcribed from DNA to RNA, but are not translated into an amino acid sequence. Other genes may function as regulators of structural genes or as regulators of DNA transcription.

By “genetically modified” is meant a gene that is altered from its native state (e.g. by insertion mutation, deletion mutation, nucleic acid sequence mutation, or other mutation), or that a gene product is altered from its natural state (e.g. by delivery of a transgene that works in trans on a gene's encoded mRNA or protein, such as delivery of inhibitory RNA or delivery of a dominant negative transgene).

By “exon” is meant a region of a gene which includes sequences which are used to encode the amino acid sequence of the gene product.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one it is operatively associated with in nature.

As used herein, the term “homology” refers to the subunit sequence identity or similarity between two polymeric molecules e.g., between two nucleic acid molecules, e.g., between two DNA molecules, or two polypeptide molecules. When a subunit position in both of the two molecules is occupied by the same monomeric subunit, e.g., if a position in each of two polypeptide molecules is occupied by phenylalanine, then they are identical at that position. The homology between two sequences, most clearly defined as the % identity, is a direct function of the number of identical positions, e.g., if half (e.g., 5 positions in a polymer 10 subunits in length) of the positions in two polypeptide sequences are identical then the two sequences are 50% identical; if 70% of the positions, e.g., 7 out of 10, are matched or homologous, the two sequences share 70% identity. By way of example, the polypeptide sequences ACDEFG and ACDHIK share 50% identity and the nucleotide sequences CAATCG and CAAGAC share 50% identity.

“Homologous recombination” is the physical exchange of DNA expedited by the breakage and reunion of two non-sister chromatids. In order to undergo recombination the DNA duplexes must have complementarity. The molecular mechanism is as follows: DNA duplexes pair, homologous strands are nicked, and broken strands exchange DNA between duplexes. The region at the site of recombination is called the hybrid DNA or heteroduplex DNA. Second nicks are made in the other strand, and the second strand crosses over between duplexes. After this second crossover event the reciprocal recombinant or splice recombinant is created. The duplex of one DNA parent is covalently linked to the duplex of another DNA parent. Homologous recombination creates a stretch of heteroduplex DNA.

A “hypomorphic mutation” is a change to the genetic material (usually DNA or RNA), which can be caused by any form of genetic mutation, and causes an decrease in normal gene function without causing a complete absence of normal gene function.

The term “inbred animal” is used herein to refer to an animal that has been interbred with other similar animals of the same species in order to preserve and fix certain characteristics, or to prevent other characteristics from being introduced into the breeding population.

The term “insertional mutation” is used herein to refer the translocation of nucleic acid from one location to another location which is in the genome of an animal so that it is integrated into the genome, thereby creating a mutation in the genome. Insertional mutations can also include knocking out or knocking in of endogenous or exogenous DNA via gene trap or cassette insertion. Exogenous DNA can access the cell via electroporation or chemical transformation. If the exogenous DNA has homology with chromosomal DNA it will align itself with endogenous DNA. The exogenous DNA is then inserted or disrupts the endogenous DNA via two adjacent crossing over events, known as homologous recombination. A targeting vector can use homologous recombination for insertional mutagenesis. Insertional mutagenesis of endogenous or exogenous DNA can also be carried out via DNA transposon. The DNA transposon is a mobile element that can insert itself along with additional exogenous DNA into the genome. Insertional mutagenesis of endogenous or exogenous DNA can be carried out by retroviruses. Retroviruses have a RNA viral genome that is converted into DNA by reverse transcriptase in the cytoplasm of the infected cell. Linear retroviral DNA is transported into the nucleus, and become integrated by an enzyme called integrase. Insertional mutagenesis of endogenous or exogenous DNA can also be done by retrotransposons in which an RNA intermediate is translated into DNA by reverse transcriptase, and then inserted into the genome.

The term “gene knockdown” refers to techniques by which the expression of one or more genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knockdown organism” or “knockdowns”.

By “knock-out” is meant an alteration in the nucleic acid sequence that reduces the biological activity of the polypeptide normally encoded there from by at least 80% compared to the unaltered gene. The alteration may be an insertion, deletion, frame shift mutation, or missense mutation. Preferably, the alteration is an insertion or deletion, or is a frame shift mutation that creates a stop codon.

An “L1 sequence” or “L1 insertion sequence” as used herein, refers to a sequence of DNA comprising an L1 element comprising a 5′ UTR, ORF1 and ORF2, a 3′ UTR and a poly A signal, wherein the 3′ UTR has DNA (e.g. a gene trap or other cassette) positioned either therein or positioned between the 3′ UTR and the poly A signal, which DNA is to be inserted into the genome of a cell.

A “mutation” is a detectable change in the genetic material in the animal, which is transmitted to the animal's progeny. A mutation is usually a change in one or more deoxyribonucleotides, the modification being obtained by, for example, adding, deleting, inverting, or substituting nucleotides. Exemplary mutations include but are not limited to a deletion mutation, an insertion mutation, a non-sense mutation or a missense mutation. Thus, the terms “mutation” or “mutated” as used herein are intended to denote an alteration in the “normal” or “wild-type” nucleotide sequence of any nucleotide sequence or region of the allele. As used herein, the terms “normal” and “wild-type” are intended to be synonymous, and to denote any nucleotide sequence typically found in nature. The terms “mutated” and “normal” are thus defined relative to one another; where a cell has two chromosomal alleles of a gene that differ in nucleotide sequence, at least one of these alleles is a “mutant” allele as that term is used herein. Based on these definitions, an “endogenous toxicology gene” is the “wild-type” gene that exists normally in a cell, and a “mutated toxicology gene” defines a gene that differs in nucleotide sequence from the wild-type gene.

“Non-homologous end joining (NHEJ)” is a cellular repair mechanism. The NHEJ pathway is defined by the ligation of blunt ended double stand DNA breaks. The pathway is initiated by double strand breaks in the DNA, and works through the ligation of DNA duplex blunt ends. The first step is recognition of double strand breaks and formation of scaffold. The trimming, filling in of single stranded overhangs to create blunt ends and joining is executed by the NHEJ pathway. An example of NHEJ is repair of a DNA cleavage site created by a zinc finger nuclease (ZFN). This would normally be expected to create a small deletion mutation.

“Nucleic Acid sequence mutation” is a mutation to the DNA of a gene that involves change of one or multiple nucleotides. A point mutation which affects a single nucleotide can result in a transition (purine to purine or pyrimidine to pyrimidine) or a transversion (purine to pyrimidine or pyrimidine to purine). A point mutation that changes a codon to represent a different amino acid is a missense mutation. Some point mutations can cause a change in amino acid so that there is a premature stop codon; these mutations are called nonsense mutations. A mutation that inserts or deletes a single base will change the entire downstream sequence and are known as frame shift mutations. Some mutations change a base pair but have no effect on amino acid representation; these are called silent mutations. Mutations to the nucleic acid of a gene can have different consequences based on their location (intron, exon, regulatory sequence, and splice joint).

“Optional” or “optionally” means that the subsequently described event or circumstance may or may not occur, and that the description includes instances where said event or circumstance occurs and instances where it does not.

The term “outbred animal” is used herein to refer to an animal that breeds with any other animal of the same species without regard to the preservation of certain characteristics.

As used herein, the term “phenotype” means any property of a cell or organism. A phenotype can simply be a change in expression of an mRNA or protein. Examples of phenotypes also include, but are in no way limited to, cellular, biochemical, histological, behavioral, or whole organismal properties that can be detected by the artisan. Phenotypes include, but are not limited to, cellular transformation, cell migration, cell morphology, cell activation, resistance or sensitivity to drugs or chemicals, resistance or sensitivity to pathogenic protein localization within the cell (e.g. translocation of a protein from the cytoplasm to the nucleus), resistance or sensitivity to ionizing radiation, profile of secreted or cell surface proteins, (e.g., bacterial or viral) infection, post-translational modifications, protein localization within the cell (e.g. translocation of a protein from the cytoplasm to the nucleus), profile of secreted or cell surface proteins, cell proliferation, signal transduction, metabolic defects or enhancements, transcriptional activity, recombination intermediate joining, DNA damage response, cell or organ transcript profiles (e.g., as detected using gene chips), apoptosis resistance or sensitivity, animal behavior, organ histology, blood chemistry, biochemical activities, gross morphological properties, life span, tumor susceptibility, weight, height/length, immune function, organ function, any disease state, and other properties known in the art. In certain situations and therefore in certain embodiments of the invention, the effects of mutation of one or more genes in a cell or organism can be determined by observing a change in one or more given phenotypes (e.g., in one or more given structural or functional features such as one or more of the phenotypes indicated above) of the mutated cell or organism compared to the same structural or functional feature(s) in a corresponding wild-type or (non-mutated) cell or organism (e.g., a cell or organism in which the gene(s) have not been mutated).

By “plasmid” is meant a circular strand of nucleic acid capable of autosomal replication in plasmid-carrying bacteria. The term includes nucleic acid which may be either DNA or RNA and may be single- or double-stranded. The plasmid of the definition may also include the sequences which correspond to a bacterial origin of replication.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

A “random site” is used herein to refer to a location in the genome where a retrotransposition or transposition or other DNA mutation event takes places, without prior intention of mutation at that particular location. It is also used herein to refer to a location in the genome that is randomly modified by any insertion mutation or deletion mutation or nucleic acid sequence mutation.

The term “regulatory sequence” is defined herein as including promoters, enhancers and other expression control elements such as polyadenylation sequences, matrix attachment sites, insulator regions for expression of multiple genes on a single construct, ribosome entry/attachment sites, introns that are able to enhance expression, and silencers.

By “reporter gene” is meant any gene which encodes a product whose expression is detectable. A reporter gene product may have one of the following attributes, without restriction: fluorescence (e.g., green fluorescent protein), enzymatic activity (e.g., lacZ or luciferase), or an ability to be specifically bound by a second molecule (e.g., biotin or an antibody-recognizable epitope).

By “retrotransposition” as used herein, is meant the process of integration of a sequence into a genome, expression of that sequence in the genome, reverse transcription of the integrated sequence to generate an extrachromosomal copy of the sequence and reintegration of the sequence into the genome.

A “retrotransposition event” is used herein to refer to the translocation of a retrotransposon from a first location to a second location with the preferable outcome being integration of a retrotransposon into the genome at the second location. The process involves a RNA intermediate, and can retrotranspose from one chromosomal location to another or from introduced exogenous DNA to endogenous chromosomal DNA.

By “selectable marker” is meant a gene product which may be selected for or against using chemical compounds, especially drugs. Selectable markers often are enzymes with an ability to metabolize the toxic drugs into non-lethal products. For example, the pac (puromycin acetyl transferase) gene product can metabolize puromycin, the dhfr gene product can metabolize trimethoprim (tmp) and the bla gene product can metabolize ampicillin (amp). Selectable markers may convert a benign drug into a toxin. For example, the HSV tk gene product can change its substrate, FIAU, into a lethal substance. Another selectable marker is one which may be utilized in both prokaryotic and eukaryotic cells. The neo gene, for example, metabolizes and neutralizes the toxic effects of the prokaryotic drug, kanamycin, as well as the eukaryotic drug, G418.

By “selectable marker gene” as used herein is meant a gene or other expression cassette which encodes a protein which facilitates identification of cells into which the selectable marker gene is inserted.

A “specific site” is used herein to refer to a location in the genome that is predetermined as the position where a retrotransposition or transposition event or other DNA mutation will take place. It is also used herein to refer to a specific location in the genome that is modified by any insertion mutation or deletion mutation or nucleic acid sequence mutation.

A “drug metabolism” gene is used herein to refer to a gene which encodes a protein that is associated with the phenotype that is characterized as altering the chemical structure or mechanism by biotransformation of a drug, xenobiotic, chemical, metabolite, environmental compound, or any other substance both endogenous and exogenous to humans, rats and other model organism. This phenotype may affect the activity, toxicity, localization, drug-drug or drug-substance interactions, or any other interaction which the substance may have within humans, rats and other model organisms. A “drug metabolism protein” is used herein to refer to a protein product of a gene that is associated with the phenotype that is characterized as altering the biotransformation of drugs, chemicals and other substances.

As used herein, the term “targeted genetic recombination” refers to a process wherein recombination occurs within a DNA target locus present in a host cell or host organism. Recombination can involve either homologous or non-homologous DNA.

The term “transfection” means the introduction of a foreign nucleic acid into a cell. The term “transformation” means the introduction of a “foreign” (i.e. extrinsic or extracellular) gene, DNA or RNA sequence to an ES cell or pronucleus, so that the cell will express the introduced gene or sequence to produce a desired substance in a genetically modified animal.

By “transgenic” is meant any animal which includes a nucleic acid sequence which is inserted by artifice into a cell and becomes a part of the genome of the animal that develops from that cell. Such a transgene may be partly or entirely heterologous to the transgenic animal. Although transgenic mice represent another embodiment of the invention, other transgenic mammals including, without limitation, transgenic rodents (for example, hamsters, guinea pigs, rabbits, and rats), and transgenic pigs, cattle, sheep, and goats are included in the definition.

By “transposition” as used herein, is meant the process of one DNA sequence insertion into another (location) without relying on sequence homology. The DNA element can be transposed from one chromosomal location to another or from introduction of exogenous DNA and inserted into the genome.

A “transposition event” or “transposon insertion sequence” is used herein to refer to the translocation of a DNA transposon either from one location on the chromosomal DNA to another or from one location on introduced exogenous DNA to another on the chromosomal DNA.

By “transposon” or “transposable element” is meant a linear strand of DNA capable of integrating into a second strand of DNA which may be linear or may be a circularized plasmid. Transposons often have target site duplications, or remnants thereof, at their extremities, and are able to integrate into similar DNA sites selected at random, or nearly random. Preferred transposons have a short (e.g., less than 300) base pair repeat at either end of the linear DNA. By “transposable elements” is meant any genetic construct including but not limited to any gene, gene fragment, or nucleic acid that can be integrated into a target DNA sequence under control of an integrating enzyme, often called a transposase.

A coding sequence is “under the control of” or “operatively associated with” transcriptional and translational control sequences in a cell when RNA polymerase transcribes the coding sequence into mRNA, which is then trans-RNA spliced (if it contains introns) and translated, in the case of mRNA, into the protein encoded by the coding sequence.

The term “variant” may also be used to indicate a modified or altered gene, DNA sequence, enzyme, cell, etc., i.e., any kind of mutant.

The term “vector” is used interchangeably with the terms “construct”, “cloning vector” and “expression vector” and means the vehicle by which a DNA or RNA sequence (e.g. a foreign gene) can be introduced into a host cell, (e.g. ES cell or pronucleus) so as to transform the host and promote expression (e.g. transcription and translation) of the introduced sequence including but not limited to plasmid, phage, transposons, retrotransposons, viral vector, and retroviral vector. By “non-viral vector” is meant any vector that does not comprise a virus or retrovirus.

A “vector sequence” as used herein, refers to a sequence of DNA comprising at least one origin of DNA replication and at least one selectable marker gene.

For the purposes of the present invention, the term “zinc finger nuclease” or “ZFN” refers to a chimeric protein molecule comprising at least one zinc finger DNA binding domain effectively linked to at least one nuclease or part of a nuclease capable of cleaving DNA when fully assembled. Ordinarily, cleavage by a ZFN at a target locus results in a double stranded break (DSB) at that locus.

The present invention provides a desired rat or a rat cell which contains a predefined, specific and desired alteration rendering the rat or rat cell predisposed to alterations in drug and chemical metabolism by modification of its structure or mechanism. Specifically, the invention pertains to a genetically altered rat, or a rat cell in culture, that is defective in at least one of two alleles of a drug metabolism gene such as the Cyp7b1 gene, the Cyp3a4 gene, etc. In one embodiment, the drug metabolism gene is the Cyp7b1 gene. In another embodiment, the drug metabolism gene is one of several known drug metabolism genes selected from the group consisting of Cyp1a1, Cyp1a2, Cyp1b1, aCyp2A, Cyp2a6, Cyp2a7, Cyp2a7p1, Cyp2a13, Cyp2b, Cyp2b6, Cyp2b7p1, Cyp2c8, Cyp2c9, Cyp2c18, Cyp2c19, Cyp2d6, Cyp2d7p1, Cyp2d7p2, Cyp2d8p1, Cyp2d8p2, Cyp2e1, Cyp2f1, Cyp2f1p, C yp2g1p, Cyp2g2p, Cyp2j2, Cyp2r1, Cyp2s1, Cyp2t2p, Cyp2t3p, Cyp2u1, Cy p2w1, Cyp3a, Cyp3a4, Cyp3a5, Cyp3a5p1, Cyp3a5p2, Cyp3a7, Cyp3a43, C yp4a11, Cyp4a22, Cyp4b1, Cyp4f2, Cyp4f3, Cyp4f3lP, Cyp4f8, Cyp4f11, Cy p4f12, Cyp4f22, Cyp4v2, Cyp4x1, Cyp4z1, Cyp4z2p, Cyp7a1, Cyp7b1, Cyp8 b1, Cyp11a1, Cyp11b1, Cyp11b2, Cyp17a1, Cyp19a1, Cyp20a1, Cyp21a1p, Cyp21a2, Cyp24a1, Cyp26a1, Cyp26b1, Cyp26c1, Cyp27a1, Cyp27b1, Cy p27c1, Cyp39a1, Cyp46a1, Cyp51a1, Cyp51p1, Cyp51p2, Ptgis, and Tbxas. In another embodiment, the rat cell is a somatic cell.

The inactivation of at least one of these drug metabolism alleles results in an animal with a higher susceptibility to altered drug and chemical metabolism. In one embodiment, the genetically altered animal is a rat of this type and is able to serve as a useful model for altered drug and chemical metabolism or toxicology and as a test animal for autoimmune and other studies. The invention additionally pertains to the use of such rats or rat cells, and their progeny in research and medicine.

In one embodiment, the invention provides a genetically modified or chimeric rat cell whose genome comprises two chromosomal alleles of a drug metabolism gene (especially, the Cyp7b1 gene), wherein at least one of the two alleles contains a mutation, or the progeny of this cell. The invention includes the embodiment of the above animal cell, wherein one of the alleles expresses a normal drug metabolism gene product. The invention includes the embodiment wherein the rat cell is a pluripotent cell such as an embryonic cell, embryonic stem (ES) cell, induced pluripotent stem cell (iPS), or spermatogonial stem (SS) cell, and in particular, wherein the drug metabolism gene is the gene. In another embodiment, the drug metabolism gene is one of several known drug metabolism genes selected from the group consisting of Cyp1a1, Cyp1a2, Cyp1b1, aCyp2A, Cyp2a6, Cyp2a7, Cyp2a7p1, Cyp2a13, Cyp2b, Cyp2b6, Cyp2b7p1, Cyp2c8, Cyp2c9, Cyp2c18, Cyp2c19, Cyp2d6, Cyp2d7p1, Cyp2d7p2, Cyp2d8p1, Cyp2d8p2, Cyp2e1, Cyp2f1, Cyp2f1p, C yp2g1p, Cyp2g2p, Cyp2j2, Cyp2r1, Cyp2s1, Cyp2t2p, Cyp2t3p, Cyp2u1, Cy p2w1, Cyp3a, Cyp3a4, Cyp3a5, Cyp3a5p1, Cyp3a5p2, Cyp3a7, Cyp3a43, C yp4a11, Cyp4a22, Cyp4b1, Cyp4f2, Cyp4f3, Cyp4f3lP, Cyp4f8, Cyp4f11, Cy p4f12, Cyp4f22, Cyp4v2, Cyp4x1, Cyp4z1, Cyp4z2p, Cyp7a1, Cyp7b1, Cyp8b1, Cyp11a1, Cyp11b1, Cyp11b2, Cyp17a1, Cyp19a1, Cyp20a1, Cyp21a1p, Cyp21a2, Cyp24a1, Cyp26a1, Cyp26b1, Cyp26c1, Cyp27a1, Cyp27b1, Cy p27c1, Cyp39a1, Cyp46a1, Cyp51a1, Cyp51p1, Cyp51p2, Ptgis, and Tbxas. In another embodiment, the rat cell is a somatic cell.

The methods of the present invention can be used to mutate any eukaryotic cell, including, but not limited to, haploid (in the case of multiple gene mutations), diploid, triploid, tetraploid, or aneuploid. In one embodiment, the cell is diploid. Cells in which the methods of the present invention can be advantageously used include, but are not limited to, primary cells (e.g., cells that have been explanted directly from a donor organism) or secondary cells (e.g., primary cells that have been grown and that have divided for some period of time in vitro, e.g., for 10-100 generations). Such primary or secondary cells can be derived from multi-cellular organisms, or single-celled organisms. The cells used in accordance with the invention include normal cells, terminally differentiated cells, or immortalized cells (including cell lines, which can be normal, established or transformed), and can be differentiated (e.g., somatic cells or germ cells) or undifferentiated (e.g., multipotent, pluripotent or totipotent stem cells).

A variety of cells isolated from the above-referenced tissues, or obtained from other sources (e.g., commercial sources or cell banks), can be used in accordance with the invention. Non-limiting examples of such cells include somatic cells such as immune cells (T-cells, B-cells, Natural Killer (NK) cells), blood cells (erythrocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, thymic nurse cells, Schwann cells, etc.). Eukaryotic germ cells (spermatocytes and oocytes) can also be used in accordance with the invention, as can the progenitors, precursors and stem cells that give rise to the above-described somatic and germ cells. These cells, tissues and organs can be normal, or they can be pathological such as those involved in diseases or physical disorders, including but not limited to immune related diseases, chronic inflammation, autoimmune responses, infectious diseases (caused by bacteria, fungi or yeast, viruses (including HIV) or parasites), in genetic or biochemical pathologies (e.g., cystic fibrosis, hemophilia, Alzheimer's disease, schizophrenia, muscular dystrophy, multiple sclerosis, etc.), or in carcinogenesis and other cancer-related processes. Rat pluripotent cells, including embryonic cells, spermatogonial stem cells, embryonic stem cells, and iPS cells are envisioned. Rat somatic cells are also envisioned.

In certain embodiments of the invention, cells can be mutated within the organism or within the native environment as in tissue explants (e.g., in vivo or in situ). Alternatively, tissues or cells isolated from the organism using art-known methods and genes can be mutated according to the present methods. The tissues or cells are either maintained in culture (e.g., in vitro), or re-implanted into a tissue or organism (e.g., ex vivo).

The invention also includes a non-human genetically modified or chimeric rat whose genome comprises two chromosomal alleles of a drug metabolism gene, wherein at least one of the two alleles contains a mutation, or the progeny of the animal, or an ancestor of the animal, at an embryonic stage (preferably the one-cell, or fertilized oocyte stage, and generally, not later than about the 8-cell stage) contains a mutation. The invention also includes the embodiment wherein the drug metabolism gene of the rat is the Cyp7b1 gene. In another embodiment, the drug metabolism gene is one of several known drug metabolism genes selected from the group consisting of Cyp1a1, Cyp1a2, Cyp1b1, aCyp2A, Cyp2a6, Cyp2a7, Cyp2a7p1, Cyp2a13, Cyp2b, Cyp2b6, Cyp2b7p1, Cyp2c8, Cyp2c9, Cyp2c18, Cyp2c19, Cyp2d6, Cyp2d7p1, Cyp2d7p2, Cyp2d8p1, Cyp2d8p2, Cyp2e1, Cyp2f1, Cyp2f1p, C yp2g1p, Cyp2g2p, Cyp2j2, Cyp2r1, Cyp2s1, Cyp2t2p, Cyp2t3p, Cyp2u1, Cy p2w1, Cyp3a, Cyp3a4, Cyp3a5, Cyp3a5p1, Cyp3a5p2, Cyp3a7, Cyp3a43, C yp4a11, Cyp4a22, Cyp4b1, Cyp4f2, Cyp4f3, Cyp4f3lP, Cyp4f8, Cyp4f11, Cy p4f12, Cyp4f22, Cyp4v2, Cyp4x1, Cyp4z1, Cyp4z2p, Cyp7a1, Cyp7b1, Cyp8 b1, Cyp11a1, Cyp11b1, Cyp11b2, Cyp17a1, Cyp19a1, Cyp20a1, Cyp21a1p, Cyp21a2, Cyp24a1, Cyp26a1, Cyp26b1,Cyp26c1, Cyp27a1, Cyp27b1, Cy p27c1, Cyp39a1, Cyp46a1, Cyp51a1, Cyp51p1, Cyp51p2, Ptgis, and Tbxas. In another embodiment, the rat cell is a somatic cell. The invention is also directed to the embodiment wherein the animal cell is a rat pluripotent cell. The invention is also directed to the embodiment wherein the animal cell is a rat somatic cell.

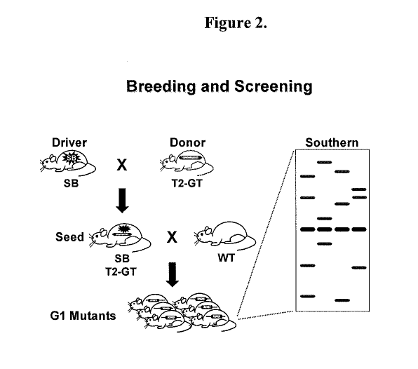

In one embodiment, the drug metabolism gene is mutated directly in the germ cells of a living organism. The separate transgenes for DNA transposon flanking ends and transposase are facilitated to create an active DNA transposon which integrates into the rat's genome. A plasmid containing transposon inverted repeats is used to create the transgenic “donor” rat. A plasmid containing transposase is used to create a separate transgenic “driver” rat. The donor rat is then bred with the driver rat to produce a rat which contains both donor transposon with flanking repeats and driver transposase (FIG. 2). This rat known as the “seed” rat has an activated DNA transposase which drives transposition events. The seed rat is bred to wild type rats to create heterozygote progeny with new transposon insertions. The heterozygotes can be interbred to create homozygous rats. Transposon insertion mutations are identified and recovered via a cloning and sequencing strategy involving the transposon-cellular DNA junction fragments. The rats that are identified to have a new DNA transposon insertion in a known gene or EST or DNA sequence of interest are called knockout rats.

In one embodiment, the drug metabolism gene is mutated in the oocyte before fusion of the pronuclei. This method for genetic modification of rats uses microinjected DNA into the male pronucleus before nuclear fusion. The microinjected DNA creates a genetically modified founder rat. A female rat is mated and the fertilized eggs are flushed from their oviducts. After entry of the sperm into the egg, the male and female pronuclei are separate entities until nuclear fusion occurs. The male pronucleus is larger are can be identified via dissecting microscope. The egg can be held in place by micromanipulation using a holding pipette. The male pronucleus is then microinjected with DNA that can be genetically modified. The microinjected eggs are then implanted into a surrogate pseudopregnant female which was mated with a vasectomized male for uterus preparation. The foster mother gives birth to genetically modified animal. The microinjection method can introduce genetic modifications directly to the germline of a living animal.

In another embodiment, the drug metabolism gene is mutated in a pluripotent cell. These pluripotent cells can proliferate in cell culture and be genetically modified without affecting their ability to differentiate into other cell types including germline cells. Genetically modified pluripotent cells from a donor can be microinjected into a recipient blastocyst, or in the case of spermatogonial stem cells can be injected into the rete testis of a recipient animal. Recipient genetically modified blastocysts are implanted into pseudopregnant surrogate females. The progeny which have a genetic modification to the germline can then be established, and lines homozygous for the genetic modification can be produced by interbreeding.

In another embodiment, the drug metabolism gene is mutated in a somatic cell and then used to create a genetically modified animal by somatic cell nuclear transfer. Somatic cell nuclear transfer uses embryonic, fetal, or adult donor cells which are isolated, cultured, and/or modified to establish a cell line. Individual donor cells are fused to an enucleated oocyte. The fused cells are cultured to blastocyst stage, and then transplanted into the uterus of a pseudopregnant female.

In one embodiment, the present invention is directed to methods for mutating a single gene or multiple genes (e.g., two or more) in eukaryotic cells and multicellular organisms. The present invention contemplates several methods for creating mutations in the drug metabolism gene(s). In one embodiment the mutation is an insertion mutation. In another embodiment the mutation is a deletion mutation. In another embodiment the method of mutation is the introduction of a cassette or gene trap by recombination. In another embodiment a small nucleic acid sequence change is created by mutagenesis (through the creation of frame shifts, stop mutations, substitution mutations, small insertion mutations, small deletion mutations, and the like). In yet another embodiment, a transgene is delivered to knockout or knockdown the products of the drug metabolism gene (mRNA or protein) in trans.

The invention also is directed to insertional mutagens for making the mutant cells and organisms, and which also can be used to analyze the mutations that are made in the cells and organisms. The invention also is directed to methods in which one or more mutated genes is tagged by a tag provided by the insertional mutagen to allow the detection, selection, isolation, and manipulation of a cell with a genome tagged by the insertional mutagen and allows the identification and isolation of the mutated gene(s). The invention provides methods for making multiple mutations (i.e., mutations in two or more genes that produce a phenotype cumulatively) in cells and organisms and tagging at least one of the mutated genes such that the mutation can be rapidly identified.

The term gene disruption as used herein refers to a gene knock-out or knock-down in which an insertional mutagen is integrated into an endogenous gene thereby resulting expression of a fusion transcript between endogenous exons and sequences in the insertional mutagen.

In one embodiment, the invention provides for insertional mutagenesis involving the integration of one or more polynucleotide sequences into the genome of a cell or organism to mutate one or more endogenous genes in the cell or organism. Thus, the insertional mutagenic polynucleotides of the present invention are designed to mutate one or more endogenous genes when the polynucleotides integrate into the genome of the cell.

Accordingly, the insertional mutagens used in the present invention can comprise any nucleotide sequence capable of altering gene expression levels or activity of a gene product upon insertion into DNA that contains the gene. The insertional mutagens can be any polynucleotide, including DNA and RNA, or hybrids of DNA and RNA, and can be single-stranded or double-stranded, naturally occurring or non-naturally occurring (e.g., phosphorothioate, peptide-nucleic acids, etc.). The insertional mutagens can be of any geometry, including but not limited to linear, circular, coiled, supercoiled, branched, hairpin, and the like, and can be any length capable of facilitating mutation, and tagging of an endogenous gene. In certain embodiments, the insertional mutagens can comprise one or more nucleotide sequences that provide a desired function.

In another embodiment, the method further involves transforming a cell with a nucleic acid construct comprising donor DNA. An example of donor DNA may include a DNA transposon. Transposable elements are discrete sequences in the genome which are mobile. They have the ability to translocate from one position in the genome to another. Unlike most genetic entities that can create modification to an organism's genome, transposons do not require homology with the recipient genome for insertion. Transposons contain inverted terminal repeats which are recognized by the protein transposase. Transposase facilitates the transposition event. Transposition can occur in replicative (the element is duplicated) or nonreplicative (element moves from one site to another and is conserved) mechanism. Transposons can either contain their own transposase or transposase can be added in trans to facilitate transposition. The transposon promotes genetic modifications in many ways. The insertion itself may cause genetic modification by disruption of a DNA sequence or introduction of DNA. The transposon may be used to deliver a gene trap.

In another embodiment, the method for mutagenesis involves transforming a cell with nucleic acid by use of a LTR retrotransposon with reverse transcriptase. The retrotransposon is initially composed of a single strand of RNA. This single stranded RNA is converted into a double stranded DNA by reverse transcriptase. This is a linear duplex of DNA that is integrated into the host's genome by the enzyme integrase. This insertion event is much like a transposition event and can be engineered to genetically modify a host's genome.

In another embodiment, the method for mutagenesis is a non-LTR retrotransposon. Long Interspersed Nucleotide Elements (LINES) are retrotransposons that do not have long terminal repeats (LTR's). The LINES open reading frame 1 (ORF1) is a DNA binding protein; ORF2 provides both reverse transcriptase and endonuclease activity. The endonucleolytic nick provides the 3′-OH end required for priming the synthesis of cDNA on the RNA template by reverse transcriptase. A second cleavage site opens the other strand of DNA. The RNA/DNA hybrid integrates into the host genome before or after converting into double stranded DNA. The integration process is called target primed reverse transcription (TPRT).

In another embodiment a retrovirus may be used for insertional genetic modification. The retroviral vector (e.g. lentivirus) inserts itself into the genome. The vector can carry a transgene or can be used for insertional mutagenesis. The infected embryos are then injected into a receptive female. The female gives birth to founder animals which have genetic modifications in their germline. Genetically modified lines are established with these founder animals.

In another embodiment, mutagenesis by recombination of a cassette into the genome may be facilitated by targeting constructs or homologous recombination vectors. Homologous recombination vectors are composed of fragments of DNA which are homologous to target DNA. Recombination between identical sequences in the vector and chromosomal DNA will result in genetic modification. The vector may also contain a selection method (e.g., antibiotic resistance or GFP) and a unique restriction enzyme site used for further genetic modification. The targeting vector will insert into the genome at a position (e.g., exon, intron, regulatory element) and create genetic modification.

In another embodiment, mutagenesis through recombination of a cassette into the genome may be carried out by Serine and Tyrosine recombinase with the addition of an insertion cassette. Site-specific recombination occurs by recombinase protein recognition of DNA, cleavage and rejoining as a phosphodiesterase bond between the serine or tyrosine residues. A cassette of exogenous or endogenous DNA may be recombined into the serine or tyrosine site. The cassette can contain a transgene, gene trap, reporter gene or other exogenous or endogenous DNA.

In one embodiment, the present invention is directed to methods for both targeted (site-specific) DNA insertions and targeted DNA deletions. In one embodiment, the method involves transformation of a cell with a nucleic acid or mRNA construct minimally comprising DNA encoding a chimeric zinc finger nuclease (ZFN), which can be used to create a DNA deletion. In another embodiment, a second DNA construct can be provided that will serve as a template for repair of the cleavage site by homologous recombination. In this embodiment, a

DNA insertion may be created. The DNA insertion may contain a gene trap cassette.

The invention also is directed to nucleic acid sequence mutation for making the mutant cells and organisms.

In one embodiment, the method involves chemical mutagenesis with mutagens such as methane-sulfonic acid ethylester (EMS), N-ethyl-N-nitrosourea (ENU), diepoxyoctane and UV/trimethylpsorlalen to create nucleic acid sequence mutations.

In another embodiment, sequence editing methods are used that involve the delivery of small DNA fragments, hybrid DNA/RNA molecules, and modified DNA polymers to create sequence mismatches and nucleic acid mutations. RNA/DNA hybrids are molecules composed of a central stretch of DNA flanked by short RNA sequences that form hairpin structures. The RNA/DNA hybrids can produce single base-pair substitutions and deletions resulting in nucleotide mutations. Some other sequence editing examples include triplex forming oligonucleotides, small fragment homologous replacement, single-stranded DNA oligonucleotides, and adeno-associated virus (AAV) vectors.

The invention also is directed to genetic expression modification or mutagenesis, which may be carried out by delivery of a transgene that works in trans.

In one embodiment, RNA interference (RNAi) may be used to alter the expression of a gene. Single stranded mRNA can be regulated by the presence of sections of double stranded RNA (dsRNA) or small interfering RNA (siRNA). Both anti-sense and sense RNAs can be effective in inhibiting gene expression. siRNA mediates RNA interference and is created by cleavage of long dsDNA by the enzyme Dicer. RNAi can create genetic modification by triggering the degradation of mRNA's that are complementary to either strand of short dsRNA. When siRNA is associated with complementary single-stranded RNA it can signal for nuclease to degrade the mRNA. RNAi can also result in RNA silencing which occurs when the short dsRNA inhibits expression of a gene. Other forms of inhibitory RNA, such as small hairpin RNA (shRNA) are envisioned.

In another embodiment, the delivery of a transgene encoding a dominant negative protein may alter the expression of a target gene. Dominant negative proteins can inhibit the activity of an endogenous protein. One example is the expression a protein which contains the ligand binding site of an endogenous protein. The expressed dominant-negative protein “soaks up” all of the available ligand. The endogenous protein is therefore not activated, and the wild type function is knocked out or knocked down.

Other schemes based on these general concepts are within the scope and spirit of the invention, and are readily apparent to those skilled in the art.

The invention also provides methods for making homozygous mutations in rats by breeding a genetically modified rat which is heterozygous for a mutant allele with another genetically modified rat which is heterozygous for the same mutant allele. On average 25% of offspring of such matings are expected to produce animals that are homozygous for the mutant allele. Homozygous mutations are useful for discovering functions associated with the mutated gene.

The present invention is directed generally to reduction or inactivation of gene function or gene expression in cells in vitro and in multicellular organisms. The invention encompasses methods for mutating cells using one or more mutagens, particularly wherein at least one mutation is an insertion mutation, a deletion mutation, or a nucleic acid sequence mutation, to achieve a homozygous gene mutation or mutation of multiple genes required cumulatively to achieve a phenotype. The methods are used to create knock-outs, knock-downs, and other modifications in the same cell or organism.

The mutation can result in a change in the expression level of a gene or level of activity of a gene product. Activity encompasses all functions of a gene product, e.g. structural, enzymatic, catalytic, allosteric, and signaling. In one embodiment, mutation results in a decrease or elimination of gene expression levels (RNA and/or protein) or a decrease or elimination of gene product activity (RNA and/or protein). Most mutations will decrease the activity of mutated genes. However, both the insertional and physicochemical mutagens can also act to increase or to qualitatively change (e.g., altered substrate on binding specificity, or regulation of protein activity) the activity of the product of the mutated gene. Although mutations will often generate phenotypes that may be difficult to detect, most phenotypically detectable mutations change the level or activity of mutated genes in ways that are deleterious to the cell or organism.

As used herein, decrease means that a given gene has been mutated such that the level of gene expression or level of activity of a gene product in a cell or organism is reduced from that observed in the wild-type or non-mutated cell or organism. This is often accomplished by reducing the amount of mRNA produced from transcription of a gene, or by mutating the mRNA or protein produced from the gene such that the expression product is less abundant or less active.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a rat. Also disclosed are animals produced by the process of transfecting a cell within the animal any of the nucleic acid molecules disclosed herein, wherein the mammal is a rat.

Such methods are used to achieve mutation of a single gene to achieve a desired phenotype as well as mutation of multiple genes, required cumulatively to achieve a desired phenotype, in a rat cell or rat. The invention is also directed to methods of identifying one or more mutated genes, made by the methods of the invention, in rat cells and in rats, by means of a tagging property provided by the insertional mutagen(s). The insertional mutagen thus allows identification of one or more genes that are mutated by insertion of the insertional mutagen.

The invention is also directed to rat cells and rats created by the methods of the invention and uses of the rat cells and rats. The invention is also directed to libraries of rat cells created by the methods of the invention and uses of the libraries.

Drug toxicology, altered drug and chemical metabolism-associated genes

The invention also features a novel genetically modified rat with a genetically engineered modification in a gene encoding a drug metabolism-associated protein. In another aspect, the invention features a genetically modified rat, wherein a gene encoding drug metabolism protein is modified resulting in reduced drug metabolism protein activity. In preferred embodiments of this aspect, the genetically modified rat is homozygous for the modified gene. In other preferred embodiments, the gene encoding the drug metabolism protein is modified by disruption, and the genetically modified rat has reduced drug metabolism protein activity. In yet another embodiment, the transgenic rat is heterozygous for the gene modification.

In another embodiment of this aspect of the invention, the invention features a nucleic acid vector comprising nucleic acid capable of undergoing homologous recombination with an endogenous drug metabolism gene in a cell, wherein the homologous recombination results in a modification of the drug metabolism gene resulting in decreased drug metabolism protein activity in the cell. In another aspect, the modification of the drug metabolism gene is a disruption in the coding sequence of the endogenous drug metabolism gene.

Another embodiment of this aspect of the invention features a rat cell, wherein the endogenous gene encoding drug metabolism protein is modified, resulting in reduced drug metabolism protein activity in the cell.

In certain embodiments, the reduced drug metabolism protein activity is manifested. In a related aspect, the invention features a rat cell containing an endogenous drug metabolism gene into which there is integrated a transposon comprising DNA encoding a gene trap and/or a selectable marker.

In another aspect, the invention features a rat cell containing an endogenous drug metabolism gene into which there is integrated a retrotransposon comprising DNA encoding a gene trap and/or a selectable marker. In another aspect, the invention features a rat cell containing an endogenous drug metabolism gene into which there is DNA comprising an insertion mutation in the drug metabolism gene. In another aspect, the invention features a rat cell containing an endogenous drug metabolism gene into which there is DNA comprising a deletion mutation in the drug metabolism gene. In another aspect, the invention features a rat cell containing an endogenous drug metabolism gene in which there has been nucleic acid sequence modification of the drug metabolism gene.

In another embodiment of the invention, the invention features a method for determining whether a compound is potentially useful for treating or alleviating the symptoms of a drug metabolism gene disorder, which includes (a) providing a cell that produces a drug metabolism protein, (b) contacting the cell with the compound, and (c) monitoring the activity of the drug metabolism protein, such that a change in activity in response to the compound indicates that the compound is potentially useful for treating or alleviating the symptoms of a drug metabolism gene disorder.

It is understood that simultaneous targeting of more than one gene may be utilized for the development of “knock-out rats” (i.e., rats lacking the expression of a targeted gene product), “knock-in rats” (i.e., rats expressing a fusion protein or a protein encoded by a gene exogenous to the targeted locus), “knock down rats” (i.e., rats with a reduced expression of a targeted gene product), or rats with a targeted gene such that a truncated gene product is expressed.

Rat models that have been genetically modified to alter drug metabolism gene expression may be used in in vivo assays to test for activity of a candidate drug metabolism modulating agent, or to further assess the role of drug metabolism gene in a drug metabolism pathway process such as T lymphocyte mediated apoptosis or native DNA autoantibody production. Preferably, the altered drug metabolism gene expression results in a detectable phenotype, such as decreased levels of P450 expression, bioavailability of a drug, increased susceptibility to toxicity, organ sequestration, compared to control animals having normal drug metabolism gene expression. The genetically modified rat may additionally have altered drug metabolism gene expression (e.g. drug metabolism gene knockout). In one embodiment, the genetically modified rats are genetically modified animals having a heterologous nucleic acid sequence present as an extrachromosomal element in a portion of its cells, i.e. mosaic animals (see, for example, techniques described by Jakobovits, 1994, Curr. Biol. 4:761-763) or stably integrated into its germ line DNA (i.e., in the genomic sequence of most or all of its cells). Heterologous nucleic acid is introduced into the germ line of such genetically modified animals by genetic manipulation of, for example, embryos or germ cells or germ cells precursors of the host animal.

Methods of making genetically modified rodents are well-known in the art (see Brinster et al., Proc. Nat. Acad. Sci. USA 82: 4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No. 4,945,050, by Sandford et al.; for genetically modified Drosophila see Rubin and Spradling, Science (1982) 218:348-53 and U.S. Pat. No. 4,670,388; for genetically modified insects see Berghammer A. J. et al., A Universal Marker for Genetically modified Insects (1999) Nature 402:370-371; for genetically modified Zebrafish see Lin S., Genetically modified Zebrafish, Methods Mol Biol. (2000); 136:375-3830); for microinjection procedures for fish, amphibian eggs and birds see Houdebine and Chourrout, Experientia (1991) 47:897-905; Hammer et al., Cell (1990) 63:1099-1112; and for culturing of embryonic stem (ES) cells and the subsequent production of genetically modified animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman genetically modified animals can be produced according to available methods (see Wilmut, I. et al. (1997) Nature 385:810-813; and PCT International Publication Nos. WO 97/07668 and WO 97/07669).

In one embodiment, the genetically modified rat is a “knock-out” animal having a heterozygous or homozygous alteration in the sequence of an endogenous drug metabolism gene that results in a dysregulation of immune function, preferably such that drug metabolism gene expression is undetectable or insignificant. Knock-out animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the genetically modified host species. For example, a mouse drug transporter gene is used to construct a homologous recombination vector suitable for altering an endogenous drug metabolism gene in the mouse genome. Detailed methodologies for homologous recombination in rodents are available (see Capecchi, Science (1989) 244:1288-1292; Joyner et al., Nature (1989) 338:153-156). Procedures for the production of non-rodent genetically modified mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as rats harboring a knockout of a specific gene, may be used to produce antibodies against the human counterpart of the gene that has been knocked out (Claesson M H et al., (1994) Scan J Immunol 40:257-264; Declerck P J et al., (1995) J Biol Chem. 270:8397-400).

In another embodiment, the genetically modified rat is a “knock-down” animal having an alteration in its genome that results in altered expression (e.g., decreased expression) of the drug metabolism gene, e.g., by introduction of mutations to the drug metabolism gene, or by operatively inserting a regulatory sequence that provides for altered expression of an endogenous copy of the drug metabolism gene.

Genetically modified rats can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a cre/loxP recombinase system is used to regulate expression of the transgene, animals containing transgenes encoding both the Cre recombinase and a selected protein are required. Such animals can be provided through the construction of “double” genetically modified animals, e.g., by mating two genetically modified animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified rats can be used in genetic studies to further elucidate the drug metabolism function pathways, as animal models of disease and disorders implicating dysregulated drug metabolism function, and for in vivo testing of candidate therapeutic agents, such as those identified in screens described below. The candidate therapeutic agents are administered to a genetically modified animal having altered drug metabolism function and phenotypic changes are compared with appropriate control animals such as genetically modified animals that receive placebo treatment, and/or animals with unaltered drug metabolism function that receive candidate therapeutic agent.

The invention also features novel genetically modified animals with a genetically engineered modification in the gene encoding drug metabolism proteins. In one aspect, the invention features a genetically modified non-human mammal, wherein a gene encoding a drug metabolism gene is provided as follows:

Steroid dehydroepianderosterone (DHEA) metabolism, bile acid synthesis from cholesterol, involvement in disease and metabolism: Cytochrome P450. Family 7, subfamily b, polypeptide 1 (Cyp7b1).

The Cyp7b1 gene encodes a protein Cytochrome P450, family 7, subfamily b, polypeptide 1. Cyp7b1 is an endoplasmic reticulum membrane protein responsible for the synthesis of primary bile acids from cholesterol via the acidic pathway. CYP7B 1 catalyzes the 7α-hydroxylation of oxysterols and 3β-hydroxysteroids such as Dehydroepianderosterone (DHEA). Transient transfection and ligand binding assays determined that 7-alpha-hydroxy-DHEA (7HD) activates androgen receptors (AR), ER-alpha and beta. These studies confirm Cyp7b1 involvement in steroid action. DHEA is the abounding adrenal steroid and a precursor of both androgens, estrogens, and other immune-regulating hormones (IRH) that lack androgenic and estrogenic activity. Therefore, the hydroxylation of DHEA by CYP7B 1 is important for many physiological functions. Organ function, especially in the brain and liver, is dependent on hormonal control. Steroids such as brain-active “neurosteroids” and steroids in other organs such as the liver are subject to local metabolism and affect the function of drugs, chemicals, and other molecules. DHEA has been implicated in a positive link to cognitive aging and has demonstrated memory enhancing properties in rodents. The DHEA level in primates declines with age; this decrease in activity is involved in age related complications such as Alzheimer's. However, DHEA replacement therapy has not been particularly successful in alleviating age related complications. A major factor in the failure of DHEA replacement therapy is it bioavailability due to local metabolism. Cyp7b1 directly controls the metabolism of DHEA and is considered a major factor in this therapeutic steroids efficacy. Cyp7b1 effect on steroid, bile acid synthesis, DHEA metabolism and that of therapeutic molecules and compounds is essential for proper metabolic activity in humans. One human with a homozygous mutation in Cyp7b1 exhibited severe cholestatic liver disease, cirrhosis, and liver synthetic failure. This phenotype was associated with elevated urinary bile excretion, the absence of primary bile acids, and a 27-hydroxycholesterol level of 4,500 times higher than normal humans. The absence of proper metabolism when Cyp7b1 is disrupted demonstrates the ability for this cytochrome P450 encoding gene to determine the efficacy and toxicity of many therapeutic molecules and compounds.

Inducible and repressible drug and chemical structure metabolism, accurate drug responsiveness prediction: Cyp3a

The Cyp3a gene family is responsible for the oxidative metabolism of a vast array of drug compounds and chemical structures. One member the Cyp3a4 gene is the most abundantly expressed P450 in the adult human liver and the enterocytes that line the small bowel. Altered expression or activity of Cyp3a4 is a reliable predictor of drug responsiveness and toxicity. Cyp3a4 expression is known to be induced and repressed by many chemical structures, and has been found to be under control of numerous transcription factors. Nuclear receptor mediated response to drugs by Cyp3a4 is largely coordinated by pregnane X receptor (PXR), constitutive androstane receptor (CAR), and hepatocyte nuclear factor-4-alpha (HNF4A). The PXR-CYP3A pathway has also been shown to exhibit control on hormone and endocrine chemical effects on different forms of cancer drug metabolism by CYP3A4 is inhibited enough by just one glass of grapefruit juice to increase the oral bioavailability of many known xenobiotics. There are many well characterized drug interactions which effect the expression of Cyp3a4 and the metabolism, absorption, clearance and toxicity of many drugs, drug metabolism, O-deethylation of phenacetin: Cyp1a2Cyp1a2 encodes a P450 drug metabolism protein which is highly expressed in the liver and facilitates the O-deethylation of phenacetin. The enzyme encoded by this gene metabolizes many compounds which are relevant to the study of pharmacology, toxicology, and carcinogenesis. The primary contribution of Cyp1a2 is protection against chemical insult. Cyp1a2 is known to metabolize more than 20 clinical drugs, and foreign chemicals such as carcinogens. The CYP1A2 protein catalyzes a major step in caffeine transformation, 3-demethylation. The expression and activity of Cyp1a2 in humans is highly variable with more than 40-fold differences being found in human liver samples. Due to this variability in gene expression, drug metabolism also fluctuates in human populations. Caffeine half-life values range from 1.5-9.5 hours. In human populations around 5-10% exhibit a polymorphism in Cyp1a2 which decreases its ability to catalyze phenacetin O-deethylation. The altered expression of the Cyp1a2 gene is relevant for the study of individualized drug responsiveness, toxicology, pharmacology, and carcinogenesis. Nitrosamine metabolism, acetaminophen bioactivation and hepatotoxicity: Cytochrome P450, Subfamily 2E1 (Cyp2e1). The protein encoded by Cyp2e1 localizes to the endoplasmic reticulum. The Cyp2e family is ethanol inducible. The Cyp2e1 gene is also inducible by other low molecular weight substrates; it is inducible in the diabetic state, and during starvation. The enzyme metabolizes endogenous substrates, such as ethanol, acetone, and acetal. CYP2E1 is critical for the transformation of ethanol to acetaldehyde and to acetate. Cyp2e1, the chief element of the microsomal ethanol oxidizing pathway contains polymorphisms in human populations. Polymorphic Cyp2e1 alleles have been associated with acetaldehyde accumulation in the liver; which can lead to alcohol liver disease. CYP2E1 substrates include therapeutic drugs and chemical compounds such as acetaminophen, isoiazid, reseratrol and exogenous substances benzene, carbon tetrachloride, ethylene glycol, and nitrosamines. Many CYP2E1 substrates are involved in pharmacology, toxicity, are established carcinogens or suspected carcinogens. CYP2E1 bioactivates the common analgesic and antipyretic acetaminophen (paracetamol) to N-acetyl-p-benzoquinone imine. Acetaminophen is used worldwide as a substitute for acetylsalicylic or aspirin. Acetaminophen causes hepatotoxicity at low frequency. However, Cyp2e1 biotransformation of acetaminophen to N-acetyl-p-benzoquinoneimine allows this metabolite to react with nucleophiles. Conjugation of N-acetyl-p-benzoquinoneimine with glutathione causes hepatotoxicity. Therefore, an increased expression of Cyp2e1 predisposes a cell or animal to hepatotoxicity from acetaminophen exposure. Cyp2e1 is therefore, a suitable gene to study toxicity, drug metabolism, pharmacology, and carcinogenesis.

The invention also features novel genetically modified cells and animals with a genetically engineered modification in a gene encoding for a drug metabolism protein. In one aspect, the invention features genetically modified rat cells or rats, wherein a gene modification occurs in a gene encoding a drug metabolism protein provided in Table 1:

TABLE 1

| Transporter | | Rat Chromo- |
|---|---|---|
| gene | Function | somal Location |
| Cyp7b1 | Catalyzes hydroxylation of oxysterols | 2q24 |
| | including DHEA. Is essential for the | |
| | predominant route of DHEA metabolism. | |
| | Implements 7alpha-hydroxylation of 27- | |
| | hydroxycholesterol in a bile synthesis | |
| | pathway. | |
| Cyp3a | The Cyp3a proteins oxidatively metabolize | 7q21-q22.1 |
| | many drugs, and have an induced response | |
| | to many drugs. Key predictors of drug | |
| | responsiveness and toxicity. | |
| Cyp1a2 | Major role is protection from chemical | 8q24 |
| | insult. O-deethylation of phenacetin, | |
| | metabolism of multiple drugs, Cyp1a2 | |
| | expression alters drug plasma half-life and | |
| | elimination, plays a key role in the organ | |
| | sequestration of multiple drugs. | |
| Cyp2e1 | Ethanol and low molecular weight | 1: (200918521- |
| | compound inducible, and metabolizer of | 200928919) bp |
| | multiple chemicals, drugs, toxins, | |
| | biotransformation of acetaminophen leads | |
| | to increased hepatotoxicity. | |

Methods

The methods used in the present invention are comprised of a combination of genetic introduction methods, genetic modification or mutagenesis mechanisms, and vector delivery methods. For all genetic modification or mutagenesis mechanisms one or more introduction and delivery method may be employed. The invention may include but is not limited to the methods described below.

Genetic Introduction Methods

In one introduction method, the drug metabolism gene is mutated directly in the germ cells of an adult animal. This method usually involves the creation of a transgenic founder animal by pronuclear injection. Rat oocytes are microinjected with DNA into the male pronucleus before nuclear fusion. The microinjected DNA creates a transgenic founder rat. In this method, a female rat is mated and the fertilized eggs are flushed from their oviducts. After entry of the sperm into the egg, the male and female pronuclei are separate entities until nuclear fusion occurs. The male pronucleus is larger are can be identified via dissecting microscope. The egg can be held in place by micromanipulation using a holding pipette. The male pronucleus is then microinjected with DNA that can be genetically modified. The microinjected eggs are then implanted into a surrogate pseudopregnant female which was mated with a vasectomized male for uterus preparation. The foster mother gives birth to transgenic founder animals. If the transgenic DNA encodes the appropriate components of a mutagenesis system, such as transposase and a DNA transposon, then mutagenesis will occur directly in the germ cells of founder animals and some offspring will contain new mutations. Chemical mutagenesis can also be used to cause direct germ line mutations.

In another introduction method, the drug metabolism gene is mutated in the early embryo of a developing animal. The mutant embryonic cells develop to constitute the germ cells of the organism, thereby creating a stable and heritable mutation. Several forms of mutagenesis mechanisms can be introduced this way including, but not limited to, zinc finger nucleases and delivery of gene traps by a retrovirus.

In another introduction method, the drug metabolism gene is mutated in a pluripotent cell. These pluripotent cells can proliferate in cell culture and be genetically modified without affecting their ability to differentiate into other cell types including germ line cells. Genetically modified pluripotent cells from a donor can be microinjected into a recipient blastocyst, or in the case of spermatogonia) stem cells can be injected into the rete testis of a recipient animal. Recipient genetically modified blastocysts are implanted into pseudopregnant surrogate females. The progeny which have a genetic modification to the germ line can then be established, and lines homozygous for the genetic modification can be produced by interbreeding.

In another introduction method, the drug metabolism gene is mutated in a somatic cell and then used to create a genetically modified animal by somatic cell nuclear transfer. Somatic cell nuclear transfer uses embryonic, fetal, or adult donor cells which are isolated, cultured, and/or modified to establish a cell line. Individual donor cells are fused to an enucleated oocyte. The fused cells are cultured to blastocyst stage, and then transplanted into the uterus of a pseudopregnant female. Alternatively the nucleus of the donor cell can be injected directly into the enucleated oocyte. See U.S. Appl. Publ. No. 20070209083.

Genetic modification methods

Mobile DNA technology

DNA transposons are discrete mobile DNA segments that are common constituents of plasmid, virus, and bacterial chromosomes. These elements are detected by their ability to transpose self-encoded phenotypic traits from one replicon to another, or to transpose into a known gene and inactivate it. Transposons, or transposable elements, include a piece of nucleic acid bounded by repeat sequences. Active transposons encode enzymes (transposases) that facilitate the insertion of the nucleic acid into DNA sequences.

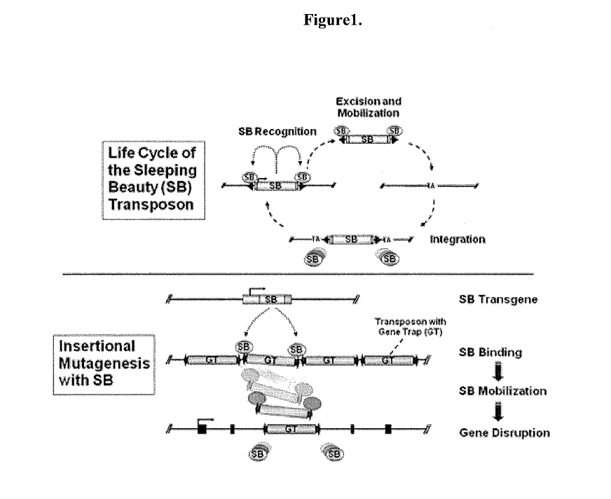

The lifecycle and insertional mutagenesis of DNA transposon Sleeping Beauty (SB) is depicted in FIG. 1. In its lifecycle, the SB encodes a transposase protein. That transposase recognizes the inverted terminal repeats (ITRs) that flank the SB transposon. The transposase then excises SB and reintegrates it into another region of the genome. Mutagenesis via Sleeping Beauty is depicted. The mechanism is similar to the life cycle, but transposase is not encoded by the transposon, but instead is encoded elsewhere in the genome

The Sleeping Beauty (SB) mutagenesis breeding and screening scheme is depicted in FIG. 2. One rat referred to as the “driver” rat contains the (SB) transposase within its genome. A second rat, the “donor” rat contains the transposon which has the transposase-recognizable inverted terminal repeats (ITRs). The two rats are bred to create the “seed” rat which has an active transposon containing transposase and ITRs. The transposon recognizes the ITRs, excises the transposon, and inserts it elsewhere in the rat's genome. This insertion event often disrupts coding, regulatory, and other functional regions in the genome to create knockout rat models. The “seed” rat is bred with wild type rats which beget heterozygous G1 mutants. If the transposon has inserted into the genome, the event will be recorded via size comparison of DNA by Southern blot analysis. The exact location of the transposon insertion is determined by PCR-based amplification methods combined with sequencing of the DNA flanking the new insertion.

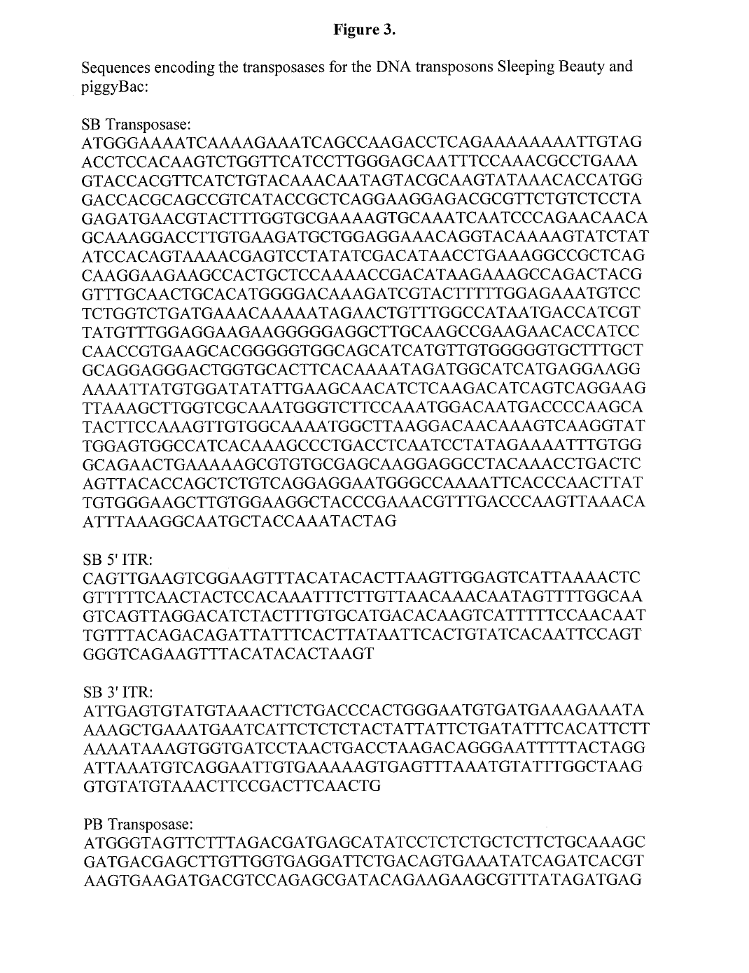

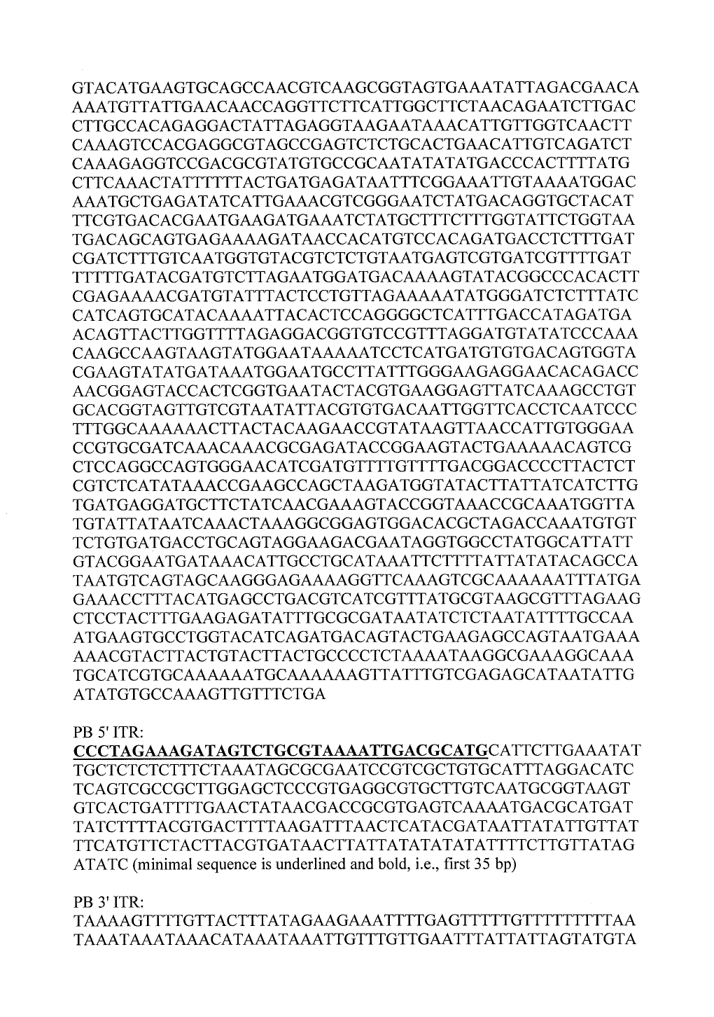

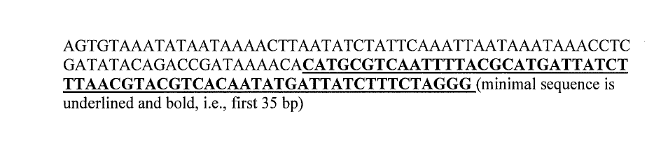

The sequences for the DNA transposons Sleeping Beauty (SB) piggyBac (PB) functional domains are shown in FIG. 3. The SB and PB transposase sequences encode the protein that recognizes the ITRs and carries out the excision and re-integration. The 3′ and 5′ ITRs are the flanking sequences which the respective transposases recognizes in order to carry out excision and reintegration elsewhere in the genome.

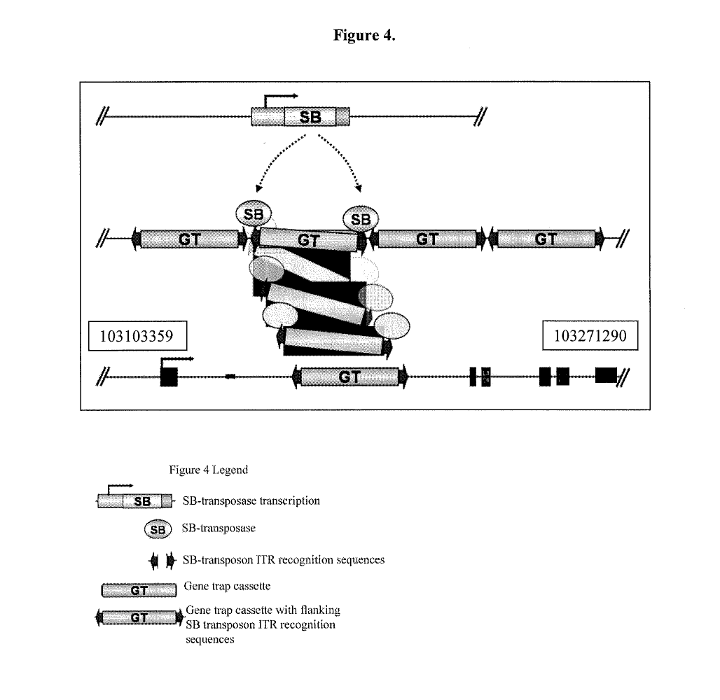

The DNA transposon Sleeping Beauty (SB) was used by the inventors to create a knockout rat in the Cyp7b1 gene. The mechanism is depicted in FIG. 4, and is the same as that described above. The transposase is encoded, and the protein recognizes the ITRs of the transposon. The transposon is then excised and reinserted into the first intron of the rat Cyp7b1 gene which resides on chromosome 13, location 13q22.

In another embodiment, the present invention utilizes the transposon piggyBac, and sequence configurations outside of piggyBac, for use as a mobile genetic element as described in U.S. Pat. No. 6,962,810. The Lepidopteran transposon piggyBac is capable of moving within the genomes of a wide variety of species, and is gaining prominence as a useful gene transduction vector. The transposon structure includes a complex repeat configuration consisting of an internal repeat (IR), a spacer, and a terminal repeat (TR) at both ends, and a single open reading frame encoding a transposase.

The Lepidopteran transposable element piggyBac transposes via a unique cut-and-paste mechanism, inserting exclusively at 5′ TTAA 3′ target sites that are duplicated upon insertion, and excising precisely, leaving no footprint (Elick et al., 1996b; Fraser et al., 1996; Wang and Fraser 1993).

In another embodiment, the present invention utilizes the Sleeping Beauty transposon system for genome manipulation as described, for example, in U.S. Pat. No. 7,148,203. In one embodiment, the system utilizes synthetic, salmonid-type Tc1-like transposases with recognition sites that facilitate transposition. The transposase binds to two binding-sites within the inverted repeats of salmonid elements, and appears to be substrate-specific, which could prevent cross-mobilization between closely related subfamilies of fish elements.

In another aspect of this invention, the invention relates to a transposon gene transfer system to introduce DNA into the DNA of a cell comprising: a nucleic acid fragment comprising a nucleic acid sequence positioned between at least two inverted repeats wherein the inverted repeats can bind to a SB protein and wherein the nucleic acid fragment is capable of integrating into DNA of a cell; and a transposase or nucleic acid encoding a transposase. In one embodiment, the transposase is provided to the cell as a protein and in another the transposase is provided to the cell as nucleic acid. In one embodiment the nucleic acid is RNA and in another the nucleic acid is DNA. In yet another embodiment, the nucleic acid encoding the transposase is integrated into the genome of the cell. The nucleic acid fragment can be part of a plasmid or a recombinant viral vector. Preferably, the nucleic acid sequence comprises at least a portion of an open reading frame and also preferably, the nucleic acid sequence comprises at least a regulatory region of a gene. In one embodiment the regulatory region is a transcriptional regulatory region and the regulatory region is selected from the group consisting of a promoter, an enhancer, a silencer, a locus-control region, and a border element. In another embodiment, the nucleic acid sequence comprises a promoter operably linked to at least a portion of an open reading frame.

In the transgene flanked by the terminal repeats, the terminal repeats can be derived from one or more known transposons. Examples of transposons include, but are not limited to the following: Sleeping Beauty (Izsvak Z, Ivies Z. and Plasterk R H. (2000) Sleeping Beauty, a wide host-range transposon vector for genetic transformation in vertebrates. J. Mol. Biol. 302:93-102), mos1 (Bessereau J L, et al. (2001) Mobilization of a Drosophila transposon in the Caenorhabditis elegans germ line. Nature. 413(6851):70-4; Zhang L, et al. (2001) DNA-binding activity and subunit interaction of the mariner transposase. Nucleic Acids Res. 29(17):3566-75, piggyBac (Tamura T. et al. Germ line transformation of the silkworm Bombyx mori L. using a piggyBac transposon-derived vector. Nat Biotechnol. 2000 January; 18(1):81-4), Himar1 (Lampe D J, et al. (1998) Factors affecting transposition of the Himar1 mariner transposon in vitro. Genetics. 149(11):179-87), Hermes, To12 element, Pokey, Tn5 (Bhasin A, et al. (2000) Characterization of a Tn5 pre-cleavage synaptic complex. J Mol Biol 302:49-63), Tn7 (Kuduvalli P N, Rao J E, Craig N L. (2001) Target DNA structure plays a critical role in Tn7 transposition. EMBO J 20:924-932), Tn916 (Marra D, Scott J R. (1999) Regulation of excision of the conjugative transposon Tn916. Mol Microbiol 2:609-621), Tc1/mariner (Izsvak Z, Ivies Z4 Hackett P B. (1995) Characterization of a Tc1-like transposable element in zebrafish (Danio rerio). Mol. Gen. Genet. 247:312-322), Minos and S elements (Franz G and Savakis C. (1991) Minos, a new transposable element from Drosophila hydei, is a member of the Tc1-like family of transposons. Nucl. Acids Res. 19:6646; Merriman P J, Grimes C D, Ambroziak J, Hackett D A, Skinner P, and Simmons M J. (1995) S elements: a family of Tc1-like transposons in the genome of Drosophila melanogaster. Genetics 141:1425-1438), Quetzal elements (Ke Z, Grossman G L, Cornel A J, Collins F H. (1996) Quetzal: a transposon of the Tc1 family in the mosquito Anopheles albimanus. Genetica 98:141-147); Txr elements (Lam W L, Seo P, Robison K, Virk S, and Gilbert W. (1996) Discovery of amphibian Tc1-like transposon families. J Mol Biol 257:359-366), Tc1-like transposon subfamilies (Ivies Z, Izsvak Z, Minter A, Hackett P B. (1996) Identification of functional domains and evolution of Tc1-like transposable elements. Proc. Natl. Acad Sci USA 93: 5008-5013), Tc3 (Tu Z. Shao H. (2002) Intra- and inter-specific diversity of Tc-3 like transposons in nematodes and insects and implications for their evolution and transposition. Gene 282:133-142), ICESt1 (Burrus V et al. (2002) The ICESt1 element of Streptococcus thermophilus belongs to a large family of integrative and conjugative elements that exchange modules and change their specificity of integration. Plasmid. 48(2): 77-97), maT, and P-element (Rubin G M and Spradling A C. (1983) Vectors for P element-mediated gene transfer in Drosophila. Nucleic Acids Res. 11:6341-6351). These references are incorporated herein by reference in their entirety for their teaching of the sequences and uses of transposons and transposon ITRs.

Translocation of Sleeping Beauty (SB) transposon requires specific binding of SB transposase to inverted terminal repeats (ITRs) of about 230 by at each end of the transposon, which is followed by a cut-and-paste transfer of the transposon into a target DNA sequence. The ITRs contain two imperfect direct repeats (DRs) of about 32 bp. The outer DRs are at the extreme ends of the transposon whereas the inner DRs are located inside the transposon, 165-166 by from the outer DRs. Cui et al. (J. Mol Biol 318:1221-1235) investigated the roles of the DR elements in transposition. Within the 1286-bp element, the essential regions are contained in the intervals bounded by coordinates 229-586, 735-765, and 939-1066, numbering in base pairs from the extreme 5′ end of the element. These regions may contain sequences that are necessary for transposase binding or that are needed to maintain proper spacing between binding sites.

Transposons are bracketed by terminal inverted repeats that contain binding sites for the transposase. Elements of the IR/R subgroup of the Tc1/mariner superfamily have a pair of transposase-binding sites at the ends of the 200-250 by long inverted repeats (IRs) (Izsvak, et al. 1995). The binding sites contain short, 15-20 by direct repeats (DRs). This characteristic structure can be found in several elements from evolutionarily distant species, such as Minos and S elements in flies (Franz and Savakis, 1991; Merriman et al, 1995), Quetzal elements in mosquitoes (Ke et al, 1996), Txr elements in frogs (Lam et al, 1996) and at least three Tc1-like transposon subfamilies in fish (Ivies et al., 1996), including SB [Sleeping Beauty] and are herein incorporated by reference.

Whereas Tc1 transposons require one binding site for their transposase in each IR, Sleeping Beauty requires two direct repeat (DR) binding sites within each IR, and is therefore classified with Tc3 in an IR/DR subgroup of the Tc1/mariner superfamily (96,97). Sleeping Beauty transposes into TA dinucleotide sites and leaves the Tc1/mariner characteristic footprint, i.e., duplication of the TA, upon excision. The non-viral plasmid vector contains the transgene that is flanked by IR/DR sequences, which act as the binding sites for the transposase. The catalytically active transposase may be expressed from a separate (trans) or same (cis) plasmid system. The transposase binds to the IR/DRs, catalyzes the excision of the flanked transgene, and mediates its integration into the target host genome.

Naturally occurring mobile genetic elements, known as retrotransposons, are also candidates for gene transfer vehicles. This mutagenesis method generally involves the delivery of a gene trap.

Retrotransposons are naturally occurring DNA elements which are found in cells from almost all species of animals, plants and bacteria which have been examined to date. They are capable of being expressed in cells, can be reverse transcribed into an extrachromosomal element and reintegrate into another site in the same genome from which they originated.

Retrotransposons may be grouped into two classes, the retrovirus-like LTR retrotransposons, and the non-LTR elements such as human L1 elements, Neurospora TAD elements (Kinsey, 1990, Genetics 126:317-326), I factors from Drosophila (Bucheton et al., 1984, Cell 38:153-163), and R2Bm from Bombyx mori (Luan et al., 1993, Cell 72: 595-605). These two types of retrotransposon are structurally different and also retrotranspose using radically different mechanisms.

Unlike the LTR retrotransposons, non-LTR elements (also called polyA elements) lack LTRs and instead end with polyA or A-rich sequences. The LTR retrotransposition mechanism is relatively well-understood; in contrast, the mechanism of retrotransposition by non-LTR retrotransposons has just begun to be elucidated (Luan and Eickbush, 1995, Mol. Cell. Biol. 15:3882-3891; Luan et al., 1993, Cell 72:595-605). Non-LTR retrotransposons can be subdivided into sequence-specific and non-sequence-specific types. L1 is of the latter type being found to be inserted in a scattered manner in all human, mouse and other mammalian chromosomes.

Some human L1 elements (also known as a LINEs) can retrotranspose (express, cleave their target site, and reverse transcribe their own RNA using the cleaved target site as a primer) into new sites in the human genome, leading to genetic disorders.

Further included in the invention are DNAs which are useful for the generation of mutations in a cell. The mutations created are useful for assessing the frequency with which selected cells undergo insertional mutagenesis for the generation of genetically modified animals and the like. Engineered L1 elements can also be used as retrotransposon mutagens. Sequences can be introduced into the L1 that increases its mutagenic potential or facilitates the cloning of the interrupted gene. DNA sequences useful for this application of the invention include marker DNAs, such as GFP, that are specifically engineered to integrate into genomic DNA at sites which are near to the endogenous genes of the host organism. Other potentially useful DNAs for delivery are regulatory DNA elements, such as promoter sequences, enhancer sequences, retroviral LTR elements and repressors and silencers. In addition, genes which are developmentally regulated are useful in the invention.

Viral Mutagenesis Methods

Viral vectors are often created using a replication defective virus vector with a genome that is partially replaced by the genetic material of interest (e.g., gene trap, selectable marker, and/or a therapeutic gene). The viral vector is produced by using a helper virus to provide some of the viral components that were deleted in the replication defective virus, which results in an infectious recombinant virus whose genome encodes the genetic material of interest. Viral vectors can be used to introduce an insertion mutation into the rat's genome. Integration of the viral genetic material is often carried out by the viral enzyme integrase. Integrase brings the ends of viral DNA together and converts the blunt ends into recessed ends. Integrase creates staggered ends on chromosomal DNA. The recessed ends of the viral DNA are then joined with the overhangs of genomic DNA, and the single-stranded regions are repaired by cellular mechanisms. Some recombinant virus vectors are equipped with cell uptake, endosomal escape, nuclear import, and expression mechanisms allowing the genetic material of interest to be inserted and expressed in the rat's genome. The genetic material introduced via viral vectors can genetically modify the rat's genome but is not limited to disrupting a gene, inserting a gene to be expressed, and by delivery of interfering RNA. Viral vectors can be used in multiple methods of delivery. The most common mode of delivery is the microinjection of a replication deficient viral vector (e.g. retroviral, adenoviral) into an early embryo (1-4 day) or a one cell pronuclear egg. After viral vector delivery, the embryo is cultured in vitro and transferred to recipient rats to create genetically modified progeny.

In one embodiment, insertion mutations can be created by delivery of a gene trap vector into the rat genome. The gene trap vector consists of a cassette that contains selectable reporter tags. Upstream from this cassette is a 3′ splice acceptor sequence. Downstream from the cassette lays a termination sequence poly adenine repeat tail (polyA). The splice accepter sequence allows the gene trap vector to be spliced into chromosomal mRNA. The polyA tail signals the premature interruption of the transcription. The result is a truncated mRNA molecule that has decreased function or is completely non-functional. The gene trap method can also be utilized to introduce exogenous DNA into the genome.

In another embodiment an enhancer trap is used for insertional mutagenesis. An enhancer trap is a transposable element vector that carries a weak minimal promoter which controls a reporter gene. When the transposable element is inserted the promoter drives expression of the reporter gene. The expression of the reporter gene also displays the expression patterns of endogenous genes. Enhancer trapping results in genetic modification and can be used for gain-of-function genetics. The Ga14-mediated expression system is an example of an enhancer trap.

Further included are one or more selectable marker genes. Examples of suitable prokaryotic marker genes include, but are not limited to, the ampicillin resistance gene, the kanamycin resistance gene, the gene encoding resistance to chloramphenicol, the lacZ gene and the like. Examples of suitable eukaryotic marker genes include, but are not limited to, the hygromycin resistance gene, the green fluorescent protein (GFP) gene, the neomycin resistance gene, the zeomycin gene, modified cell surface receptors, the extracellular portion of the IgG receptor, composite markers such as beta-geo (a lac/neo fusion) and the like.

In one embodiment, the gene trap will need to be integrated into the host genome and an integrating enzyme is needed. Integrating enzymes can be any enzyme with integrating capabilities. Such enzymes are well known in the art and can include but are not limited to transposases, integrases, recombinases, including but not limited to tyrosine site-specific recombinases and other site-specific recombinases (e.g., cre), bacteriophage integrases, retrotransposases, and retroviral integrases.

The integrating enzymes of the present invention can be any enzyme with integrating capabilities. Such enzymes are well known in the art and can include but are not limited to transposases (especially DDE transposases), integrases, tyrosine site-specific recombinases and other site-specific recombinases (e.g., cre), bacteriophage integrases, integrons, retrotransposases, retroviral integrases and terminases.

Disclosed are compositions, wherein the integrating enzyme is a transposase. It is understood and herein contemplated that the transposase of the composition is not limited and to any one transposase and can be selected from at least the group consisting of Sleeping Beauty (SB), Tn7, Tn5, mos1, piggyBac, Himar1, Hermes, To12, Pokey, Minos, S elements, P-elements, ICESt1, Quetzal elements, Tn916, maT, Tc1/mariner and Tc3.

Where the integrating enzyme is a transposase, it is understood that the transposase of the composition is not limited and to any one transposase and can be selected from at least the group consisting of Sleeping Beauty (SB), Tn7, Tn5, Tn916, Tc1/mariner, Minos and S elements, Quetzal elements, Txr elements, maT, mos1, piggyBac, Himar1, Hermes, To12, Pokey, P-elements, and Tc3. Additional transposases may be found throughout the art, for example, U.S. Pat. No. 6,225,121, U.S. Pat. No. 6,218,185 U.S. Pat. No. 5,792,924 U.S. Pat. No. 5,719,055, U.S. Patent Application No. 20020028513, and U.S. Patent Application No. 20020016975 and are herein incorporated by reference in their entirety. Since the applicable principal of the invention remains the same, the compositions of the invention can include transposases not yet identified.

Also disclosed are integrating enzymes of the disclosed compositions wherein the enzyme is an integrase. For example, the integrating enzyme can be a bacteriophage integrase. Such integrase can include any bacteriophage integrase and can include but is not limited to lamda bacteriophage and mu bacteriophage, as well as Hong Kong 022 (Cheng Q., et al. Specificity determinants for bacteriophage Hong Kong 022 integrase: analysis of mutants with relaxed core-binding specificities. (2000) Mol Microbiol. 36(2):424-36.), HP1 (Hickman, A. B., et al. (1997). Molecular organization in site-specific recombination: The catalytic domain of bacteriophage HP1 integrase at 2.7 A resolution. Cell 89: 227-237), P4 (Shoemaker, N B, et al. (1996). The Bacteroides mobilizable insertion element, NBU1, integrates into the 3′ end of a Leu-tRNA gene and has an integrase that is a member of the lambda integrase family. J Bacteriol. 178(12):3594-600.), P1 (Li Y, and Austin S. (2002) The P1 plasmid in action: time-lapse photomicroscopy reveals some unexpected aspects of plasmid partition. Plasmid. 48(3):174-8.), and T7 (Rezende, L. F., et al. (2002) Essential Amino Acid Residues in the Single-stranded DNA-binding Protein of Bacteriophage T7. Identification of the Dimer Interface. J. Biol. Chem. 277, 50643-50653.). Integrase maintains its activity when fused to other proteins.

Also disclosed are integrating enzymes of the disclosed compositions wherein the enzyme is a recombinase. For example, the recombinase can be a Cre recombinase, Flp recombinase, HIN recombinase, or any other recombinase. Recombinases are well-known in the art. An extensive list of recombinases can be found in Nunes-Duby S E, et al. (1998) Nuc. Acids Res. 26(2): 391-406, which is incorporated herein in its entirety for its teachings on recombinases and their sequences.

Also disclosed are integrating enzymes of the disclosed compositions wherein the enzyme is a retrotransposase. For example, the retrotransposase can be a GATE retrotransposase (Kogan G L, et al. (2003) The GATE retrotransposon in Drosophila melanogaster: mobility in heterochromatin and aspects of its expression in germ line tissues. Mol Genet Genomics. 269(2):234-42).

Other general techniques for integration into the host genome include, for example, systems designed to promote homologous recombination. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

Zinc Finger Nucleases

In another method, a zinc finger nuclease creates site-specific deletions via double-stranded DNA breaks that are repaired by non-homologous end joining (NHEJ). Zinc finger nucleases may also be used to create an insertion mutation by combining the ZFN with a homologously integrating cassette to create an insertion in the genomic DNA. Therefore, this genetic modification method can be used for both targeted (site-specific) DNA insertions and targeted DNA deletions. In one embodiment, the method involves transformation of a cell with a nucleic acid or mRNA construct minimally comprising DNA encoding a chimeric zinc finger nuclease (ZFN), which can be used to create a DNA deletion. In another embodiment, a second DNA construct can be provided that will serve as a template for repair of the cleavage site by homologous recombination. In this embodiment, a DNA insertion may be created. The DNA insertion may contain a gene trap cassette. In one embodiment, this method can be combined with spermatogonial stem cell technology or embryonic stem cell technology, as mentioned above. In another embodiment, this method can be combined with mobile DNA technology. This technique can also be done directly in the rat embryo.

Nucleic Acid Modification Methods

In one embodiment, a random mutation is created with a chemical mutagen and then a screen is performed for insertions in a particular drug metabolism gene. Chemical mutagens such as methane-sulfonic acid ethylester (EMS), N-ethyl-N-nitrosourea (ENU), diepoxyoctane and UV/trimethylpsorlalen may be employed to create nucleic acid sequence mutations.

Sequence editing methods can also be used that involve the delivery of small DNA fragments, hybrid DNA/RNA molecules, and modified DNA polymers to create sequence mismatches and nucleic acid mutations. RNA/DNA hybrids are molecules composed of a central stretch of DNA flanked by short RNA sequences that form hairpin structures. The RNA/DNA hybrids can produce single base-pair substitutions and deletions resulting in nucleotide mutations. Some other sequence editing examples include triplex forming oligonucleotides, small fragment homologous replacement, single stranded DNA oligonucleotides, and adeno-associated virus (AAV) vectors.

The invention also is directed to genetic expression modification or mutagenesis by delivery of a transgene that works in trans.

In one genetic modification method, RNA interference may be used to alter the expression of a gene. In another genetic modification method, the delivery of a transgene encoding a dominant negative protein may alter the expression of a target gene.

Vector Delivery Methods

The mutagenesis methods of this invention may be introduced into one or more cells using any of a variety of techniques known in the art such as, but not limited to, microinjection, combining the nucleic acid fragment with lipid vesicles, such as cationic lipid vesicles, particle bombardment, electroporation, DNA condensing reagents (e.g., calcium phosphate, polylysine or polyethyleneimine) or incorporating the nucleic acid fragment into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector.

DNA or other genetic material may be delivered through viral and non-viral vectors. These vectors can carry exogenous DNA that is used to genetically modify the genome of the rat. For example Adenovirus (AdV), Adeno-associated virus (AAV), and Retrovirus (RV) which contain LTR regions flanking a gene trap, transgene, cassette or interfering RNA are used to integrate and deliver the genetic material. Another delivery method involves non-viral vectors such as plasmids used for electroporation and cationic lipids used for lipofection. The non-viral vectors usually are engineered to have mechanisms for cell uptake, endosome escape, nuclear import, and expression. An example would be a non-viral vector containing a specific nuclear localization sequence and sequence homology for recombination in a targeted region of the genome.

There are a number of compositions and methods which can be used to deliver nucleic acids to cells, either in vitro or in vivo. For example, the nucleic acids can be delivered through a number of direct delivery systems such as, electroporation, lipofection, calcium phosphate precipitation, plasmids, cosmids, or via transfer of genetic material in cells or carriers such as cationic liposomes. Appropriate means for transfection, including chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed non-viral vectors for example, lipids such as liposomes, such as cationic liposomes (e.g., DOTMA, DOPE, DC-cholesterol) or anionic liposome, or polymersomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the vector can be administered as a component of a microcapsule that can be targeted to specific cell types, such as macrophages, or where the diffusion of the compound or delivery of the compound from the microcapsule is designed for a specific rate or dosage.

In the methods described above, which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

These vectors may be targeted to a particular cell type via antibodies, receptors, or receptor ligands. The following references are examples of the use of this technology to target specific proteins to tumor tissue and are incorporated by reference herein (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid-mediated drug targeting to colonic carcinoma), receptor-mediated targeting of DNA through cell specific ligands, lymphocyte-directed tumor targeting, and highly specific therapeutic retroviral targeting of murine glioma cells in vivo. The following references are examples of the use of this technology to target specific proteins to tumor tissue and are incorporated by reference herein (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Nucleic acids that are delivered to cells which are to be integrated into the host cell genome typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Cyp7b1 domains and loss of function mutations

Rattus norvegicus Cytochrome P450 7, family B, polypeptide 1 is a 507 amino acid (AA) protein. The protein consists of a conserved CYP domain between AA: 44-487. However, there are only 3 absolutely conserved residues whose topography and fold is highly conserved. The conserved core contains a coil, a helix bundle, and two sets of beta-sheets. The core makes up the haem-binding loop (with an absolutely conserved cysteine that serves as the 5th ligand for the haem iron), the proton-transfer groove and the absolutely conserved EVLR motif in helix K. This conserved motif is located between AA 221-224 (EVLR) of the Rattus norvegicus protein. The protein also has a conserved metal binding site at position 354. The Cyp7b1 gene mRNA consists of 2626 base pairs with a coding sequence between base pairs 119-1642. A highly conserved region which is essential for proper metabolism and CYP function is 164 by in length between by 1782-1945.

Martin et al. (J. Clin. Endocr. Metab. 89:2928-2935, 2004) found that CYP7B 1 catalyzes oxysterol hydroxylation of steroids including DHEA. Setchell et al. (J. Clin. Invest. 102:1690-1703, 1998) discovered an inborn mutation in Cyp7b1 which results in AA change R388X. When homozygous, this mutation causes in severe cholestatic liver disease. This same mutation is also the cause sporadic and progressive spastic paraplegia (SPG5A), a motor neuron degenerative disease.

TABLE
AMINO Acid changes resulting in drug metabolism
pathway modification.
This table displays some amino acid changes that are predicted to
disrupt Cyp7b1 activity.

| Amino Acid | Cyp7b1 functional domain effected |
|---|---|
| 44-487 | Cytochrome P450 haem-thiolate activity |
| 221-224 | Core helix motif |
| 354 | Metal binding site |
| 388 | Bile acid synthesis defect, cholestatic liver |
| | disease, spastic paraplegia |

Cyp7b1 phenotypes

The Cyp7b1 gene encodes the protein Cytochrome P450 family 7, polypeptide 1. Cyp7b1 plays a critical role in bile acid synthesis, and steroid metabolism. These functions of Cyp7b1 affect the metabolism and functionality of therapeutic molecules such as the adrenal steroid DHEA. DHEA has been shown to display memory enhancing properties in animal models and its expression in human's decreases alongside aging and related disorders. However, DHEA replacement therapy has not been successful in humans to alleviate age related disorders largely due to the effect of DHEA metabolism by Cyp7b1. Further, in humans mutations within the Cyp7b1 gene result in severe cholestatic liver disease. In the absence of functional Cyp7b1 a bile acid synthesis defect occurs. The primary effects of cholestatic liver disease are absence of primary bile acids, and severe increase in 27-hydroxycholesterol levels. Some Cyp7b1 mutations result in partial loss of function or “knockdown” and others result in full loss of function mutations or “knockout”.

The Cyp7b1 activity resulting from a loss of function in one or several Cyp7b1 effectors has completely different and variable phenotypes; some resulting in a more subtle alteration in drug metabolism. Complete loss of function or “knockout” of Cyp7b1 resulting in loss of function in all of its effectors always results in bile synthesis defects, hydroxycholesterol level elevation, and therapeutic molecule (DHEA) metabolism defects. These defects resulting from non-functional Cyp7b1 are known to affect the metabolism of therapeutic molecules in known animal models. This therapeutic metabolism alteration effects the activation or deactivation of a drug, its clearance, and its therapeutic effectiveness. Animal models exhibiting defects in the Cyp7b1 gene are models of drug metabolism, efficacy and toxicity.

Table. Drug Metabolism Gene Phenotypes

| | Drug or chemical | |
|---|---|---|
| Gene | metabolism altered | KO phenotype 123 |
| Cyp7b1 | Dehydroepianderosterone | Mice deficient for Cyp7b1 lack proper DHEA |
| | (DHEA) | metabolism, exhibit elevated expression of |
| | | oxysterols, and have a predisposition to |
| | | cholesterol gallstone formation. |
| Cyp3a | Docetaxel (Taxotere), | Mice deficient for gene family Cyp3a |
| | Acetaminophen, | function are unable to metabolize docetaxel. |
| | Gestodene, Sulfentanil | The Cyp3a deficient mice had severe |
| | (~50% of all prescribed | increased exposure and bioavailability of |
| | drugs see: Ann. Rev. | docetaxe1 . . . |
| | Pharma Toxicol. 39: 1-7, | |
| | 1999) | |
| Cyp1a2 | Caffeine, polycyclic | Cyp1a2 encodes a gene that is involved in |
| | aromatic hydrocarbons, | drug metabolism, effects the bioavailability of |
| | aflatoxin B, | multiple compounds, and acts as an inducible |
| | acetaminophen, | hepatic binding protein involved in liver |
| | heterocyclic amines, | sequestration of drugs. |
| | carcinogenic arylamines, | |
| | theophylline, | |
| | imipramine, clozapine, | |
| | propranolol. | |
| Cyp2e1 | Ethanol, acetaldehyde, | Cyp2e1-/- mice are more resistant to |
| | acetaminophen, | acetaminophen hepatotoxicity. The mice |
| | acrylaminde, aniline, | survive much larger doses of the drug when |
| | benzene, butanol, carbon | compared to WT. |
| | tetrachloride. See Chem | |
| | Res Toxicol 4, 169 | |
| | (1991) | |

CLUSTAL 2.0.10 multiple sequence alignment of rat and mouse Solute carrier family 7,
member 11 (Cyp7b1) amino acid sequence. The sequence alignment shows close
homology between the mouse and rat Cyp7b1 sequence. The homology of conserved
domains and knowledge of insertion mutagenesis allows evidence that mutagenesis will
create a total knockout rat Cyp7b1.

| | | |
|---|---|---|
| Rattus | -----------------GCTTTGGGGGCCGCTGTGACATTCTGCTCTGCACTGCGGGCAG | 43 |
| Mus | ACTAACTTTGTAGTTCAGCTTTGGGGGCCGCTGTGACATTCTGCTCTGTGCAGCGGGCAG | 60 |
| | ******************************* * ******** | |
| Rattus | GCCAGAGCCTCTGGTCTAGAAGAGAGGGCACTTTGCAGAAGCCATCGCTCACTACAGAGC | 103 |
| Mus | GCCACAGCCTCTGGTCTAGAAGAGAGGGCACTGTGCAGAAGCCATCGCTCCCTACAGAGC | 120 |
| | **** *************************** ***************** ********* | |
| Rattus | CGCCAGAGCGTCCGGATGGAGGGAGCCACGACCCCAGATGCCGCTTCGCCCGGGCCTCTC | 163 |
| Mus | CGCCAGCTCGTCGGGATGCAGGGAGCCACGACCCTAGATGCCGCCTCGCCAGGGCCTCTC | 180 |
| | ****** **** ***** *************** ********* ***** ********* | |
| Rattus | TCCCTACTAGGCCTTCTCTTTGCGGTCACCTTGCTGCTCCCAGTCCTGTTCCTCCTCACC | 223 |
| Mus | GCCCTCCTAGGCCTTCTCTTTGCCGCCACCTTACTGCTCTCGGCCCTGTTCCTCCTTACC | 240 |
| | **** ***************** * ****** ****** * * ************ *** | |
| Rattus | CGGCGCACAAGGCGTCCTTGTGAACCTCCCTTGATAAAAGGTTGGATTCCTTATCTTGGC | 283 |
| Mus | CGGCGCACCAGGCGCCCTCGTGAACCACCCTTGATAAAAGGTTGGCTTCCTTATCTTGGC | 300 |
| | ******** ***** *** ******* ****************** ************** | |
| Rattus | ATGGCCCTGAAATTCTGGAAGGATCCGTTAGCTTTCTTGCAAACTCTTCAAAGGCAATGT | 343 |
| Mus | ATGGCCCTGAAATTCTTTAAGGATCCGTTAACTTTCTTGAAAACTCTTCAAAGGCAACAT | 360 |
| | **************** ************ ******** ***************** * | |
| Rattus | GGTGACACTTTCACTGTCTTACTTGGAGGGAAGTATATAACATTTGTTCTGAACCCTTTC | 403 |
| Mus | GGTGACACTTTCACTGTCTTCCTTGTGGGGAAGTATATAACATTTGTTCTGAACCCTTTC | 420 |
| | ******************** **** ********************************* | |
| Rattus | CAGTACCAGTATGTAATGAAAAACCCAAAACAATTAAGCTTTGAGAAGTTCAGCCGAAGA | 463 |
| Mus | CAGTACCAGTATGTAACGAAAAACCCAAAACAATTAAGCTTTCAGAAGTTCAGCAGCCGA | 480 |
| | **************** ************************* *********** * ** | |
| Rattus | TTATCAGCGAAAGCCTTCTCTGTCAAGAAGCTGCTAACTAATGACGACCTTAGCAATGAC | 523 |
| Mus | TTATCAGCGAAAGCCTTCTCTGTAAAGAAGCTGCTTACTGATGACGACCTTAATGAAGAC | 540 |
| | *********************** *********** *** ************ * *** | |
| Rattus | ATTCACAGAGGCTATCTTCTTTTACAAGGCAAATCTCTGGATGGTCTTCTGGAAACCATG | 583 |
| Mus | GTTCACAGAGCCTATCTACTTCTACAAGGCAAACCTTTGGATGCTCTTCTGGAAACTATG | 600 |
| | ********* ****** *** *********** ** ****** ************ *** | |
| Rattus | ATCCAAGAAGTAAAAGAAATATTTGAGTCCAGACTGCTAAAACTCACAGATTGGAATACA | 643 |
| Mus | ATCCAAGAAGTAAAAGAATTATTTGAGTCCCAACTGCTAAAAATCACAGATTGGAACACA | 660 |
| | ****************** *********** ********** ************* *** | |
| Rattus | GCAAGAGTATTTGATTTCTGTAGTTCACTGGTATTTGAAATCACATTTACAACTATATAT | 703 |
| Mus | GAAAGAATATTTGCATTCTGTGGCTCACTGGTATTTGAGATCACATTTGCGACTCTATAT | 720 |
| | * **** ****** ****** * ************** ********* * *** ***** | |
| Rattus | GGAAAAATTCTTGCTGCTAACAAAAAACAAATTATCAGTGAGCTGAGGGATGATTTTTTA | 763 |
| Mus | GGAAAAATTCTTGCTGGTAACAAGAAACAAATTATCAGTGAGCTAAGGGATGATTTTTTT | 780 |
| | **************** ****** ******************** ************** | |
| Rattus | AAATTTGATGACCATTTCCCATACTTAGTATCTGACATACCTATTCAGCTTCTAAGAAAT | 823 |
| Mus | AAATTTGATGACATGTTCCCATACTTAGTATCTGACATACCTATTCAGCTTCTAAGAAAT | 840 |
| | ************ ********************************************* | |
| Rattus | GCAGAATTTATGCAGAAGAAAATTATAAAATGTCTCACACCAGAAAAAGTAGCTCAGATG | 883 |
| Mus | GAAGAATCTATGCAGAAGAAAATTATAAAATGCCTCACATCAGAAAAAGTAGCTCAGATG | 900 |
| | * ***** ************************ ****** ******************** | |
| Rattus | CAAAGACGGTCAGAAATTGTTCAGGAGAGGCAGGAGATGCTGAAAAAATACTACGGGCAT | 943 |
| Mus | CAAGGACAGTCAAAAATTGTTCAGGAAAGGCAAGATCTGCTGAAAAGATACTATAGGCAT | 960 |
| | *** *** **** ************* ***** ** ********* ****** ***** | |
| Rattus | GAAGAGTTTGAAATAGGAGCACATCATCTTGGCTTGCTCTGGGCCTCTCTAGCAAACACC | 1003 |
| Mus | GACGATCCTGAAATAGGAGCACATCATCTTGGCTTTCTCTGGGCCTCTCTAGCAAACACC | 1020 |
| | ** ** *************************** ************************ | |
| Rattus | ATTCCAGCTATGTTCTGGGCAATGTATTATCTTCTTCAGCATCCAGAAGCTATGGAAGTC | 1063 |
| Mus | ATTCCAGCTATGTTCTGGGCAATGTATTATATTCTTCGGCATCCTGAAGCTATGGAAGCC | 1080 |
| | ****************************** ****** ****** ************* * | |
| Rattus | CTGCGTGACGAAATTGACAGCTTCCTGCAGTCAACAGGTCAAAAGAAAGGACCTGGAATT | 1123 |
| Mus | CTGCGTGACGAAATTGACAGTTTCCTGCAGTCAACAGGTCAAAAGAAAGGACCTGGAATT | 1140 |
| | ******************** *************************************** | |
| Rattus | TCTGTCCACTTCACCAGAGAACAATTGGACAGCTTGGTCTGCCTGGAAAGCGCTATTCTT | 1183 |
| Mus | TCAGTCCACTTCACCAGAGAACAATTGGACAGCTTGGTCTGCCTGGAAAGCACTATTCTT | 1200 |
| | ** ************************************************ ******** | |
| Rattus | GAGGTTCTGAGGTTGTGCTCCTACTCCAGCATCATCCGTGAAGTGCAAGAGGATATGGAT | 1243 |
| Mus | GAGGTTCTGAGGCTGTGCTCATACTCCAGCATCATCCGAGAAGTGCAGGAGGATATGAAT | 1260 |
| | ************ ******* ***************** ******** ********* ** | |
| Rattus | TTCAGCTCAGAGAGTAGGAGCTACCGTCTGCGGAAAGGAGACTTTGTAGCTGTCTTTCCT | 1303 |
| Mus | CTCAGCTTAGAGAGTAAGAGTTTCTCTCTGCGGAAAGGAGATTTTGTAGCCCTCTTTCCT | 1320 |
| | ****** ******** *** * * *************** ******** ******** | |
| Rattus | CCAATGATACACAATGACCCAGAAGTCTTCGATGCTCCAAAGGACTTTAGGTTTGATCGC | 1363 |
| Mus | CCACTCATACACAATGACCCGGAAATCTTCGATGCTCCAAAGGAATTTAGGTTCGATCGC | 1380 |
| | *** * ************** *** ******************* ******** ****** | |
| Rattus | TTCGTAGAAGATGGTAAGAAGAAAACAACGTTTTTCAAAGGAGGAAAAAAGCTGAAGAGT | 1423 |
| Mus | TTCATAGAAGATGGTAAGAAGAAAAGCACGTTTTTCAAAGGAGGGAAGAAGCTGAAGACT | 1440 |
| | *** ********************* ***************** ** ********** * | |
| Rattus | TACATTATACCATTTGGACTTGGAACAAGCAAATGTCCAGGCAGATACTTTGCAATTAAT | 1483 |
| Mus | TACGTTATGCCTTTTGGACTCGGAACAAGCAAATGTCCAGGGAGATATTTTGCAGTGAAC | 1500 |
| | *** **** ** ******** ******************** ***** ****** * ** | |
| Rattus | GAAATGAAGCTACTAGTGATTATACTTTTAACTTATTTTGATTTAGAAGTCATTGACACT | 1543 |
| Mus | GAAATGAAGTTACTGCTGATTATGCTTTTAACTTATTTTGATTTAGAAATTATCGACAGG | 1560 |
| | ********* **** ******* ************************ * ** **** | |
| Rattus | AAGCCTATAGGACTAAACCACAGTCGCATGTTTCTGGGCATTCAGCATCCAGACTCTGAC | 1603 |
| Mus | AAGCCTATAGGGCTAAATCACAGTCGGATGTTTTTAGGTATTCAGCACCCCGATTCTGCC | 1620 |
| | *********** ***** ******** ****** * ** ******** ** ** **** * | |
| Rattus | ATCTCATTTAGGTACAAGGCAAAATCTTGGAGATCCTGAAAGGGTGGCAGAGAAGCTTAG | 1663 |
| Mus | GTCTCCTTTAGGTACAAAGCAAAATCTTGGAGAAGCTGAAAGTGTGGCAGAGAAGCTTTG | 1680 |
| | **** *********** *************** ******* *************** * | |
| Rattus | CGGAATAAGGCTGCACATGCTGAGCTCTGTGATTTGCTGTACTCCCC-AAATGCAGCCAC | 1722 |
| Mus | CAGAGTAAGGCTGCATGTGCTGAGCTCCGTGATTTGGTGCACTCCCCCAAATGCAACCGC | 1740 |
| | * ** ********** ********** ******** ** ******* ******* ** * | |
| Rattus | TATTCTTGTTTGTTAGAAAATGGCAAATTTTTATTTGATTGCGATCCATCCAGTTTGTTT | 1782 |
| Mus | TACTCTTGTTTG----AAAATGGCAAATTTATATTTGGTTGAGATCAATCCAGTTGGTTT | 1796 |
| | ** ********* ************** ****** *** **** ******** **** | |
| Rattus | TGGGTCACAAAACCTGTCATAAAATAAAGCGCTGTCATGGTGTAAAAAAATGTCATGGCA | 1842 |
| Mus | TGGGTCACAAAACCTGTCATAAAATAAAGCAGTGTGATGGTTTAAAAAA-TGTCATGGCA | 1855 |
| | ****************************** *** ***** ******* ********** | |
| Rattus | ATCATTTCAGGATAAGGTAAAATAACGTTTTCAAGTTTGTACTTACTATGATTTTTATCA | 1902 |
| Mus | ATCATTTCAGGATAAGGTAAAATAACATTTTCAAGTTTGTACTTACTATGATTTTTATCA | 1915 |
| | ************************** ********************************* | |
| Rattus | TTTGTAGTGAATGTGCTTTTCCAGTAATAAATTTGCGCCAGGGTGATTTTTTTTAAATTA | 1962 |
| Mus | TTTGTAGTGAATGTGCTTTTCT-GTAATAAATTTGCTCCAGGGTGATTTTATTTAAGTTA | 1974 |
| | ********************* ************* ************* ***** *** | |
| Rattus | CTGAAATCCTCTAATATGGTTTTATGTGCTGCCAGAAAAGTGTGCCATCAATGGACAGTA | 2022 |
| Mus | CTGAAATCCTCTAATATGGTTTTATGTTCTGCCAGAAAGCTGTGCCTTCAATGGACAGTC | 2034 |
| | *************************** ********** ****** ************ | |
| Rattus | TAACAATTTCCAGTTTTCCAGAGAAGGGAGAAATTAAACCCCATGAGTTACGCTGTATAA | 2082 |
| Mus | TAACAATTTCCCGTTTTCCAGAGAAGGGGGACATTAAATCCCATGAATTACACTGTATGA | 2094 |
| | *********** **************** ** ****** ******* **** ****** * | |
| Rattus | AATTGTTCTCTTCAACTATAATATCAATAATGTCTATATCACCAGGTTACCTTTGCATTA | 2142 |
| Mus | AAATTTTCCCTTCAAGTATAGTATCAATAACGTCTACATCACCAGGGTACCTTTGCATTA | 2154 |
| | ** * *** ****** **** ********* ***** ********* ************* | |
| Rattus | AATGAGTTTTGCAAAAGATTAAATGTCCCAACTTCCTTTCAATATTTAATCATCCATAAA | 2202 |
| Mus | AATGAGTTTTGC------------------------------------------------ | 2166 |
| | ************ | |
| Rattus | TGCTCTTTATAACTTTGTATTCTACAGCTCAATAGAACACAATTAATTGTTATGTAAGAT | 2262 |
| Mus | ------------------------------------------------------------ | |
| Rattus | TGCTCATGTTCAAGTTAGATATTGTTAAATTTTAATGTATATGAAAACACAAAGCCTATT | 2322 |
| Mus | ------------------------------------------------------------ | |
| Rattus | CCATAGTCAGCAGTTACTCTAAACATTAGTATTAGGTTTATTGTGGAGGTAAATGTTGAA | 2382 |
| Mus | ------------------------------------------------------------ | |
| Rattus | GTTACAATAGCACTGATGGTAGAATTGGTCTGTAGTATAAACTGTGTTAGCTGATGCTGC | 2442 |
| Mus | ------------------------------------------------------------ | |
| Rattus | AATGAACCCTTTACCAAACATTTCCATTTCTATAAACAGAGAAATTAGGTAGCATTATTT | 2502 |
| Mus | ------------------------------------------------------------ | |
| Rattus | ATCCACAACCTTTTGGACTTGAAGTTAAGTTTATTATTTTAAACTATTAAATTAAACTAT | 2562 |
| Mus | ------------------------------------------------------------ | |
| Rattus | ACAAAATAATTCTATGTGATACCATATAGCCTGTATTGATTAGTAAAAGATTTCTACTAT | 2622 |
| Mus | ------------------------------------------------------------ | |
| Rattus | CAAA | 2626 |
| Mus | ---- | |

Cytochrome P450 gene knockout phenotypes.

Cytochrome P450 family7, subfamily b, polypeptide. (Cyp7b1) Knockout, complete loss of function phenotype.

Brain and liver function is subject to hormonal control due to synthesis and metabolism of steroids. An important steroid, dehydroproepiandrosterone (DHEA) has been implicated in biological phenomenon such as cognitive aging, immunosenescence, steroid and drug elimination. DHEA has also been shown to be regulated by pro-inflammatory cytokines and may contribute to maintenance of chronic inflammation in rheumatoid arthritis (RA) in humans. DHEA is also an important therapeutic molecule. DHEA treatment in animal models has shown to alleviate age related diseases and enhance cognitive memory. The proper metabolism of DHEA can have a profound effect on this drug's efficacy. Cytochrome P450 gene Cyp7b1 catalyses 7-α-hydroxylation of oxysterols and 3β-hydroxysteroids, such as DHEA. In WT mice and rats Northern blot analysis of Cyp7b1 displays expression in multiple tissues, including brain, spleen, heart, prostate, lung, ovary, kidney and liver. Biochemical analysis reveals extensive hydroxylation of 7-α-hydroxyl-DHEA (7D) in the same tissues. In order to delineate the metabolic route and contribution of Cyp7b1 to DHEA hydroxylation Lathe et al (J. Biol Chemistry (2005) 276 (26): 23937) created Cyp7b1 knockout mice. Homologous recombination of IRES-lacZ insertion/replacement targeting vector was utilized to disrupt the Cyp7b1 gene by insertion into exon 2. Northern blot confirmed that the insertion created a null mutation with no Cyp7b1 expression. In WT mice total brain extracts converted DHEA into two products which were confirmed by TLC and gas chromatography with mass spectrometry. The most abundant product was determined to be 7-α-hydroxy-DHEA (7D) and a very small amount of product was determined to be 17β-HSD. Brain extracts from heterologous animals displayed a 53% reduction in DHEA conversion. In the brain of Cyp7b−/− mice scanning radiography revealed a less than 0.1% of WT DHEA conversion rate. When other tissues, spleen, thymus, heart, lung, prostate, uterus, and mammary gland were examined in Cyp7b−/− mice; failure of conversion was observed. When the liver and kidney which play a major role in metabolism and activation of drugs were examined, DHEA conversion to 7D was significantly altered. The WT mouse brain converts A/anediol to 6α-hydroxyl-A/anediol and other minor derivatives. When Cyp7b−/− mice are studied no such conversion is revealed in multiple tissues by scanning quantification of TLC plates. The researchers concluded that Cyp7b is essential for the function of a major hepatic and extrahepatic pathway of steroid/sterol B-ring hydroxylation. In the liver hydroxylation promotes metabolic elimination which is promoted by Cyp7b. This study suggests that Cyp7b permits steroid and drug access to receptor targets and therefore is validated as an important model for toxicology studies. The metabolism of DHEA is crucial for effectiveness as a therapeutic agent, and plays an essential role in drug metabolism. Cytochrome P450 family member 3A (Cyp3a) Knockout, complete loss of function phenotypes

The human Cyp3a gene family encodes proteins which exhibit the primary metabolism of over half of prescribed medications. Since there are no clear orthologous pairs between the mouse Cyp3as and human CYP3As the combined function of all mouse Cyp3as correspond to the combined function of all human CYP3As. Therefore, the 8 full length Cyp3a genes (Cyp3a11, 3a16, 3a25, 3a31, 3a44, 3a57, 3a59, 3a13) and 3 pseudogenes (Cyp3a58ps, 3a60ps, 3a61ps) were inactivated. Cornelia et. al (J. Clin. Invest 117, 11: 3583, 2007)) created the Cyp3a family of knockouts by replacing regions of each gene or cluster of genes with targeting vectors which contained selection cassettes. The Cyp3a genes make up an important detoxification system which contributes to the metabolism of an array of drugs. One important example is the metabolism of anticancer drug docetaxel (Taxotere) which allows for the study of toxicity due to its narrow therapeutic window. In order to study Cyp3a effect on docetaxel metabolism conversion measurement to its primary M-2 metabolite was completed in microsomes and from liver and intestine. WT microsomes efficiently formed the M-2 metabolite whereas in Cyp3a−/− microsomes M-2 formation was not recognized. In order to study the effect of Cyp3a genes on exposure to docetaxel the drug was administered orally and i.v. to WT and Cyp3a−/− mice. The drug levels were monitored frequently in blood samples. After i.v. administration Cyp3a−/− exhibited 6.8 fold higher plasma docetaxel in the plasma than WT mice. After oral administration Cyp3a−/− mice displayed a 17.7-fold higher plasma level of docetaxel than in WT mice. The oral bioavailability in WT mice was measured to be 8.1% and in Cyp3a−/− mice it was measured to be 21.2%. The Cyp3a knockout models are therefore validated as powerful tools to study drug metabolism, absorption, and clearance. Cytochrome P450, family 1, subfamily a, polypeptide 2 (Cyp1a2) Knockout phenotype.

Liang et al. (PNAS 93, 4: 1671-1676, 1996) successfully disrupted the Cyp1a2 gene in mouse by targeting vector insertion. The hprt gene replaced most of exon 2 and all of exons 3-5. The region disrupted included the conserved cysteine containing peptide in N-terminus and the cytochrome P450 “conserved tridecapeptide”. The targeting vector mutation rendered the allele completely null with and absence of mRNA production. In order to study Cyp1a2 involvement in drug metabolism the muscle relaxant zoxazolamine was administered in a paralysis lest. Zoxazolamine was administered to Cyp1a2 (−/−), Cyp1a2(+/−) and Cyp1a2(−/−) mice after a Cyp1a2 inducer was given as a single dose. The Cyp1a2(+/−) mice exhibited an intermediary phenotype when compared to WT. The Cyp1a2(−/−) mice were paralyzed upon muscle relaxant administration for at least 9 times longer than the WT mice. This prolonged paralyzation phenotype displays the ability of functional Cyp1a2 to metabolize the muscle relaxant zoazolamine. This study validates the knockout model as one relevant to drug metabolism, pharmacology, toxicology, and carcinogenesis.

Emissions from machines that combust fossil fuels contain many environmental contaminants such as polyhalogenated aromatic hydrocarbons (PHAHs), the most potent being 2,3,7,8-Tetrachlorodibenzo-p-dioxin (TCDD). Humans become exposed to these contaminants through a mixture of food, water, soil, dust, smoke, and air. TCDD has been known to accumulate in the liver in a dose dependent manner. In order to study hepatic sequestration of TCDD gene targeting disruption of the mouse Cyp1a2 gene was completed as previously described (Pineau et al. Mol. Pharmacol. 36, 113(1989)). The Cyp1a2 knockout mice and WT mice were treated with TCDD. There was no liver sequestration in any of the Cyp1a2 knockout mice. The liver-to-adipose (L/F) ratios were taken from knockout and WT mice. The L/F ratio is a very sensitive measurement that reflects the concentration of chemicals in the liver and adipose. An L/F ratio of greater than 1 represents a greater concentration of chemical in the liver, and ratios less than 1 represent a greater concentration of chemical in the adipose. The L/F ratio was 0.2 for Cyp1a2−/− mice and 3.6 for WT mice. This study proves that the Cyp1a2 gene encodes an inducible hepatic binding protein. This inducible protein effects the sequestration, metabolism, toxicology and carcinogenesis of multiple compounds. This study validates the Cyp1a2 knockout mouse as a valuable model for drug metabolism. Cytochrome P450 family 2, subfamily e, polypeptide 1 (Cyp2e1) KO phenotypes.

Susanna et al. (J. Biol. Chem. 1996 (271) 20, 12063) disrupted the mouse Cyp2e1 gene by homologous integration of a targeting vector which replaced exon2 with the PGK-NEO cassette. When Cyp2e1 mRNA analysis was done transcripts were found for the gene and the insert disrupted gene in WT and heterozygous mice respectively, but no transcript was found in Cyp2e1(−/−) mice; indicating that there was no expression of the p450 protein. To study the effect of Cyp2e1 on hepatotoxicity of acetaminophen the drug was administered to WT and knockout mice. Survival curves produced clearly displayed the role of Cyp2e1 on acetaminophen hepatotoxicity. Cyp2e1−/− were more resistant to toxicity and survived doses of up to 400 mg/kg of acetaminophen; whereas over 50% of WT mice succumb to toxicity and die at the same dose. The researchers concluded that Cyp2e1 is responsible for acetaminophen hepatotoxicity. This study validates the Cyp2e1 knockout to be a valuable model for analyzing drug metabolism.

### EXAMPLES

The rat and progenies thereof of the present invention may be any rat or progenies thereof, so long as they are a rat or progenies thereof in which genome is modified so as to have decreased or deleted activity of the drug metabolism gene.

Gene Disruption Technique which Targets at a Gene Encoding Cytochrome P450, family 7, subfamily b, polypeptide 1 (Cyp7b1).

The gene disruption method may be any method, so long as it can disrupt the gene of the target enzyme. Examples include a homologous recombination method, a method using retrovirus, a method using DNA transposon, and the like.

(a) Preparation of the rat and progenies thereof of the present invention by homologous recombination

The rat and the progenies thereof of the present invention can be produced by modifying a target gene on chromosome through a homologous recombination technique which targets at a gene encoding the drug metabolism gene. The target gene on chromosome can be modified by using a method described in Gene Targeting, A Practical Approach, IRL Press at Oxford University Press (1993) (hereinafter referred to as “Gene Targeting, A Practical Approach”); or the like, for example.

Based on the nucleotide sequence of the genomic DNA, a target vector is prepared for homologous recombination of a target gene to be modified (e.g., structural gene of the drug metabolism gene, or a promoter gene). The prepared target vector is introduced into an embryonic stem cell and a cell in which homologous recombination occurred between the target gene and target vector is selected.

The selected embryonic stem cell is introduced into a fertilized egg according to a known injection chimera method or aggregation chimera method, and the embryonic stem cell-introduced fertilized egg is transplanted into an oviduct or uterus of a pseudopregnant female rat to thereby select germ line chimeras.

The selected germ line chimeras are crossed, and individuals having a chromosome into which the introduced target vector is integrated by homologous recombination with a gene region on the genome which encodes the drug metabolism protein are selected from the born offspring.

The selected individuals are crossed, and homozygotes having a chromosome into which the introduced target vector is integrated by homologous recombination with a gene region on the genome which encodes the drug metabolism protein in both homologous chromosomes are selected from the born offspring. The obtained homozygotes are crossed to obtain offspring to thereby prepare the rat and progenies thereof of the present invention.

(b) Preparation of the rat and progenies thereof of the present invention by a method using a transposon

The rat and progenies thereof of the present invention can be prepared by using a transposon system similar to that described in Nature Genet., 25, 35 (2000) or the like, and then by selecting a mutant of the drug metabolism gene.

The transposon system is a system in which a mutation is induced by randomly inserting an exogenous gene into chromosome, wherein an gene trap cassette or exogenous gene interposed between transposons is generally used as a vector for inducing a mutation, and a transposase expression vector for randomly inserting the gene into chromosome is introduced into the cell at the same time. Any transposase can be used, so long as it is suitable for the sequence of the transposon to be used. As the gene trap cassette or exogenous gene, any gene can be used, so long as it can induce a mutation in the DNA of the cell.

The rat and progenies thereof of the present invention can be prepared by introducing a mutation into a gene encoding the drug metabolism associated protein, and then by selecting a rat of interest in which the DNA is mutated.

Specifically, the method includes a method in which a rat of interest in which the mutation occurred in the gene encoding the CYP7B 1 protein is selected from mutants born from generative cells which are subjected to mutation-inducing treatment or spontaneously generated mutants. In another embodiment, the drug metabolism gene is one of several known drug metabolism genes selected from the group consisting of

Cyp1a1, Cyp1a2,Cyp1b1, aCyp2A, Cyp2a6, Cyp2a7, Cyp2a7p1, Cyp2a13, Cyp2b, Cyp2b6, Cyp2b7p1, Cyp2c8, Cyp2c9, Cyp2c18, Cyp2c19, Cyp2d6, Cyp2d7p1, Cyp2d7p2, Cyp2d8p1, Cyp2d8p2, Cyp2e1, Cyp2f1, Cyp2f1p, C yp2g1p, Cyp2g2p, Cyp2j2, Cyp2r1, Cyp2s1, Cyp2t2p, Cyp2t3p, Cyp2u1, Cy p2w1, Cyp3a, Cyp3a4, Cyp3a5, Cyp3a5p1, Cyp3a5p2, Cyp3a7, Cyp3a43, C yp4a11, Cyp4a22, Cyp4b1, Cyp4f2, Cyp4f3, Cyp4f3lP, Cyp4f8, Cyp4f11, Cy p4f12, Cyp4f22, Cyp4v2, Cyp4x1, Cyp4z1, Cyp4z2p, Cyp7a1, Cyp7b1, Cyp8b1, Cyp11a1, Cyp11b1, Cyp11b2, Cyp17a1, Cyp19a1, Cyp20a1, Cyp21a1p, Cyp21a2, Cyp24a1, Cyp26a1, Cyp26b1, Cyp26c1, Cyp27a1, Cyp27b1, Cy p27c1, Cyp39a1, Cyp46a1, Cyp51a1, Cyp51p1, Cyp51p2, Ptgis, and Tbxas. In another embodiment, the rat cell is a somatic cell. The generative cell includes cells capable of forming an individual such as a sperm, an ovum or a pluripotent cells. The generative cell may also be a somatic cell and the animal may then be created by somatic cell nuclear transfer.

Examples in which several methods described above have been employed by the inventors to create a drug metabolism gene model phenotype in Rattus norvegicus are described below.

Genetic modification to Rattus norvegicus drug metabolism gene

Solute carrier family 7, member 11 (Cyp7b]) was carried out by a DNA transposon insertional mutagenesis method similar to that described in Nature Genet., 25, 35 (2000). The DNA transposon-mediated genetically modified allele was designated Cyp7b1Tn (sb-T2/Bart3)2.237Mcwi. The mutant strain symbol for the drug metabolism rat was designated F344-Cyp7b1Tn (sbT2/Bart3)2.237Mcwi. The DNA transposon insertion occurred in chromosome 2, within intron 1 of the rat Cyp7b1 gene. The sequence tag map position was between base pairs: 103165401 103165427. The sequence tag was: TATACATGGTCCCAGGTGGCAGCCTAG

Thus, a DNA transposon was inserted into the Cyp7b1 gene of Rattus norvegicus rendering the gene completely inactive. Cytochrome P450, family 7, subfamily b, polypeptide 1 (Cyp7b1 -) KO rats are unable to metabolize dehydroepiandrosterone (DHEA) to 7α-hydroxy-DHEA. The altered enzyme activities are inherited from the disruption in the Cyp7b1 gene. This rat knockout model is a valuable tool for studying drug metabolism as the metabolism of a therapeutic steroid, DHEA, is altered to reflect the variability of DHEA metabolism in human populations.

The practice of the present invention will employ, unless otherwise indicated, conventional techniques of molecular biology (including recombinant techniques), microbiology, cell biology and biochemistry, which are within the skill of the art.

1. A genetically modified non-human mammal, or progenies thereof, at least some of whose cells comprise a genome comprising a genetic mutation in one or more genes that causes the mammal to have a greater susceptibility to drug and chemical metabolism or toxicology than a mammal not comprising the genetic mutation.

2. The genetically modified nonhuman mammal of claim 1, wherein the mammal is a chimeric mammal.

3. The genetically modified nonhuman mammal of claim 1, wherein the mammal is a rat.

4. The genetically modified nonhuman mammal of claim 3, wherein one or more toxicology genes or loci are misexpressed.

5. The genetically modified nonhuman mammal of claim 3, wherein one or more toxicology genes are conditionally misexpressed.

6. The non-human animal model of claim 4, wherein the misexpression results in decreased expression of one or more drug metabolism gene product.

7. The genetically modified nonhuman mammal of claim 4, wherein the one or more genes encoding a drug metabolism gene product is disrupted.

8. The genetically modified nonhuman mammal of claim 4, wherein all alleles on the genome of the toxicology gene are disrupted.

9. The genetically modified nonhuman mammal of claim 4, wherein the toxicology gene is selected from the group consisting of
Cyp1a1, Cyp1a2, Cyp1b1, aCyp2A, Cyp2a6, Cyp2a7, Cyp2a7p1, Cyp2a13, Cyp2b, Cyp2b6, Cy p2b7p1, Cyp2c8, Cyp2c9, Cyp2c18, Cyp2c19, Cyp2d6, Cyp2d7p1, Cyp2d7p2, Cyp2d8p1, Cyp 2d8p2, Cyp2e1, Cyp2f1, Cyp2f1p, Cyp2g1p, Cyp2g2p, Cyp2j2, Cyp2r1, Cyp2s1, Cyp2t2p, Cyp2 t3p, Cyp2u1, Cyp2w1, Cyp3a, Cyp3a4, Cyp3a5, Cyp3a5p1, Cyp3a5p2, Cyp3a7, Cyp3a43, Cyp 4a11, Cyp4a22, Cyp4b1, Cyp4f2, Cyp4f3, Cyp4f3lP, Cyp4f8, Cyp4f11, Cyp4f12, Cyp4f22, Cyp4v2, Cyp4x1, Cyp4z1, Cyp4z2p, Cyp7a1, Cyp7b1, Cyp8b1, Cyp11a1, Cyp11b1, Cyp11a2, Cyp17a1, Cyp19a1, Cyp20a1, Cyp21a1p, Cyp21a2, Cyp24a1, Cyp26a1, Cyp26b1, Cyp26c1, Cyp27a1, Cyp27b1, Cyp27c1, Cyp39a1, Cyp46a1, Cyp51a1, Cyp51p1, Cyp51p2, Ptgis,and Tbxas.

10. The genetically modified nonhuman mammal of claim 4, wherein the toxicology gene is selected from the group consisting of Cyp7b1, Cyp3a4, Cyp1a2 and Cyp2e1.

11. The genetically modified nonhuman mammal of claim 4, wherein Cyp7b1.

12. The genetically modified nonhuman mammal of claim 4, wherein the cells are somatic cells.

13. The genetically modified nonhuman mammal of claim 4, wherein the cells are hepatocytes.

14. The genetically modified nonhuman mammal of claim 4, wherein the one or more toxicology genes or loci are disrupted using a method selected from the group consisting of mutating directly in the germ cells of a living organism, removal of DNA encoding all or part of the drug transporter protein, insertion mutation, transposon insertion mutation, deletion mutation, introduction of a cassette or gene trap by recombination, chemical mutagenesis, RNA interference (RNAi), and delivery of a transgene encoding a dominant negative protein, which may alter the expression of a target gene.

15. The genetically modified nonhuman mammal of claim 7, wherein the mammal is homozygous for the one or more disrupted genes or loci.

16. The genetically modified nonhuman mammal of claim 7, wherein the mammal is heterozygous for the one or more disrupted genes or loci.

17. A genetically modified non-human mammal, or progenies thereof, whose genome is disrupted at one or more toxicology gene loci so as to produce a phenotype, relative to a wild-type phenotype, comprising abnormal altered drug metabolism function of the mammal.

18. The genetically modified nonhuman mammal of claim 16, wherein the disruption causes the mammal to have a greater susceptibility to altered drug metabolism function.

19. The genetically modified nonhuman mammal of claim 16, wherein the mammal is a rat.

20. The genetically modified nonhuman mammal of claim 16, wherein the disruption causes a complete loss-of-function phenotype.

21. The genetically modified nonhuman mammal of claim 16, wherein the disruption causes a partial loss-of-function phenotype.

22. The genetically modified nonhuman mammal of claim 16, wherein the disruption causes a phenotype resulting from multiple transporter disruptions.

23. The genetically modified nonhuman mammal of claim 16, wherein the protein product of the toxicology gene is associated with the phenotype that is characterized as altered drug metabolism function.

24. The genetically modified nonhuman mammal of claim 16, wherein the toxicology gene is selected from the group consisting of Cyp7b1, Cyp3a4, Cyp1a2 and Cyp2e1.

25. The genetically modified nonhuman mammal of claim 16, wherein Cyp7b1.

26. The genetically modified nonhuman mammal of claim 16, wherein the one or more toxicology genes or loci are disrupted by transposon insertion mutations.

27. The genetically modified nonhuman mammal of claim 16, wherein the one or more toxicology genes or loci are disrupted by deletion mutation.

28. The genetically modified nonhuman mammal of claim 16, wherein the one or more toxicology genes or loci are disrupted by the introduction of a cassette or gene trap by recombination.

29. The genetically modified nonhuman mammal of claim 16, wherein the one or more toxicology genes or loci are disrupted by chemical mutagenesis with mutagens.

30. The genetically modified nonhuman mammal of claim 16, wherein the one or more toxicology genes or loci are disrupted by RNA interference (RNAi).

31. The genetically modified nonhuman mammal of claim 16, wherein the one or more toxicology genes or loci are disrupted by delivery of a transgene encoding a dominant negative protein, which may alter the expression of a target gene.

32. The genetically modified nonhuman mammal of claim 16, wherein the mammal is homozygous for the one or more disrupted genes or loci.

33. The genetically modified nonhuman mammal of claim 16, wherein the mammal 1 is heterozygous for the one or more disrupted genes or loci.

34. The genetically modified nonhuman mammal of claim 16, wherein the phenotype results from a diminished amount, relative to the wild-type phenotype, of a protein selected from the group consisting of Cyp7b1.

35-57. (canceled)


SEQUENCE LISTING (8 sequences)

SEQ ID NO: 1 - DNA, 2564 bp, Rattus norvegicus
gctttgggggccgctgtgacattctgctctgcactgcgggcaggccagagcctctggtctagaagagagggcactttgcagaagccatcgctcactacagagccgccagagcgtccggatggagggagccacgaccccagatgccgcttcgcccgggcctctctccctactaggccttctctttgcggtcaccttgctgctcccagtcctgttcctcctcaccggcgcacaaggcgtccttgtgaacctcccttgataaaaggttggattccttatcttggcatggccctgaaattctggaaggatccgttagctttcttgcaaactcttcaaaggcaatgtggtgacactttcactgtcttacttggagggaagtatataacatttgttctgaaccctttccagtaccagtatgtaatgaaaaacccaaaacaattaagctttgagaagttcagccgaagattatcagcgaaagccttctctgtcaagaagctgctaactaatgacgaccttagcaatgacattcacagaggctatcttcttttacaaggcaaatctctggatggtcttctggaaaccatgatccaagaagtaaaagaaatatttgagtccagactgctaaaactcacagattggaatacagcaagagtatttgatttctgtagttcactggtatttgaaatcacatttacaactatatatggaaaaattcttgctgctaacaaaaaacaaattatcagtgagctgagggatgattttttaaaatttgatgaccatttcccatacttagtatctgacatacctattcagcttctaagaaatgcagaatttatgcagaagaaaattataaaatgtctcacaccagaaaaagtagctcagatgcaaagacggtcagaaattgttcaggagaggcaggagatgctgaaaaaatactacgggcatgaagagtttgaaataggagcacatcatcttggcttgctctgggcctctctagcaaacaccattccagctatgttctgggcaatgtattatcttcttcagcatccagaagctatggaagtcctgcgtgacgaaattgacagcttcctgcagtcaacaggtcaaaagaaaggacctggaatttctgtccacttcaccagagaacaattggacagcttggtctgcctggaaagcgctattcttgaggttctgaggttgtgctcctactccagcatcatccgtgaagtgcaagaggatatggatttcagctcagagagtaggagctaccgtctgcggaaaggagactttgtagctgtctttcctcaatgatacacaatgacccagaagtcttcgatgctccaaaggactttaggtttgatcgcttcgtagaagatggtaagaagaaaacaacgtttttcaaaggaggaaaaaagctgaagagttacattataccatttggacttggaacaagcaaatgtccaggcagatactttgcaattaatgaaatgaagctactagtgattatacttttaacttattttgatttagaagtcattgacactatctcatttaggtacaaggcaaaatcttggagatcctgaaagggtggcagagaagcttagcggaataaggctgcacatgctgagctctgtgatttgctgtactccccaaatgcagccactattcttgtttgttagaaaatggcaaatttttatttgattgcgatccatccagtttgttttgggtcacaaaacctgtcataaaataaagcgctgtcatggtgtaaaaaaatgtcatggcaatcatttcaggataaggtaaaataacgttttcaagtttgtacttactatgatttttatcatttgtagtgaatgtgcttttccagtaataaatttgcgccagggtgattttttttaaattactgaaatcctctaatatggttttatgtgctgccagaaaagtgtgccatcaatggacagtataacaatttccagttttccagagaagggagaaattaaaccccatgagttacgctgtataaaattgttctcttcaactataatatcaataatgtctatatcaccaggttacctttgcattaaatgagttttgcaaaagattaaatgtcccaacttcctttcaatatttaatcatccataaatgctctttataactttgtattctacagctcaatagaacacaattaattgttatgtaagattgctcatgttcaagttagatattgttaaattttaatgtatatgaaaacacaaagcctattccatagtcagcagttactctaaacattagtattaggtttattgtggaggtaaatgttgaagttacaatagcactgatggtagaattggtctgtagtataaactgtgttagctgatgctgcaatgaaccctttaccaaacatttccatttctataaacagagaaattaggtagcattatttatccacaaccttttggacttgaagttaagtttattattttaaactattaaattaaactatacaaaataattctatgtgataccatatagcctgtattgattagtaaaagatttctactatcaaa

SEQ ID NO: 2 - DNA, 2166 bp, Mus musculus
actaactttgtagttcagctttgggggccgctgtgacattctgctctgtgcagcgggcaggccacagcctctggtctagaagagagggcactgtgcagaagccatcgctccctacagagccgccagctcgtcgggatgcagggagccacgaccctagatgccgcctcgccagggcctctcgccctcctaggccttctctttgccgccaccttactgctctcggccctgttcctccttacccggcgcaccaggcgccctcgtgaaccacccttgataaaaggttggcttccttatcttggcatggccctgaaattctttaaggatccgttaactttcttgaaaactcttcaaaggcaacatggtgacactttcactgtcttccttgtggggaagtatataacatttgttctgaaccctttccagtaccagtatgtaacgaaaaacccaaaacaattaagctttcagaagttcagcagccgattatcagcgaaagccttctctgtaaagaagctgcttactgatgacgaccttaatgaagacgttcacagagcctatctacttctacaaggcaaacctttggatgctcttctggaaactatgatccaagaagtaaaagaattatttgagtcccaactgctaaaaatcacagattggaacacagaaagaatatttgcattctgtggctcactggtatttgagatcacatttgcgactctatatggaaaaattcttgctggtaacaagaaacaaattatcagtgagctaagggatgatttttttaaatttgatgacatgttcccatacttagtatctgacatacctattcagcttctaagaaatgaagaatctatgcagaagaaaattataaaatgcctcacatcagaaaaagtagctcagatgcaaggacagtcaaaaattgttcaggaaaggcaagatctgctgaaaagatactataggcatgacgatcctgaaataggagcacatcatcttggctttctctgggcctctctagcaaacaccattccagctatgttctgggcaatgtattatattcttcggcatcctgaagctatggaagccctgcgtgacgaaattgacagtttcctgcagtcaacaggtcaaaagaaaggacctggaatttcagtccacttcaccagagaacaattggacagcttggtctgcctggaaagcactattcttgaggttctgaggctgtgctcatactccagcatcatccgagaagtgcaggaggatatgaatctcagcttagagagtaagagtttctctctgcggaaaggagattttgtagccctctttcctccactcatacacaatgacccggaaatcttcgatgctccaaaggaatttaggttcgatcgcttcatagaagatggtaagaagaaaagcacgtttttcaaaggagggaagaagctgaagacttacgttatgccttttggactcggaacaagcaaatgtccagggagatattttgcagtgaacgaaatgaagttactgctgattatgcttttaacttattttgatttagaaattatcgacaggaagcctatagggctaaatcacagtcggatgtttttaggtattcagcaccccgattctgccgtctcctttaggtacaaagcaaaatcttggagaagctgaaagtgtggcagagaagctttgcagagtaaggctgcatgtgctgagctccgtgatttggtgcactcccccaaatgcaaccgctactcttgtttgaaaatggcaaatttatatttggttgagatcaatccagttggttttgggtcacaaaacctgtcataaaataaagcagtgtgatggtttaaaaaatgtcatggcaatcatttcaggataaggtaaaataacattttcaagtttgtacttactatgatttttatcatttgtagtgaatgtgcttttctgtaataaatttgctccagggtgattttatttaagttactgaaatcctctaatatggttttatgttctgccagaaagctgtgccttcaatggacagtctaacaatttcccgttttccagagaagggggacattaaatcccatgaattacactgtatgaaaattttcccttcaagtatagtatcaataacgtctacatcaccagggtacctttgcattaaatgagttttgc

SEQ ID NO: 3 - DNA, 1023 bp, Artificial Sequence
  SB Transposase
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 4 - DNA, 229 bp, Artificial Sequence
  SB 5′ ITR
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagt

SEQ ID NO: 5 - DNA, 229 bp, Artificial Sequence
  SB 3′ITR
attgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 6 - DNA, 1785 bp, Artificial Sequence
  PB Transposase
atgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctga

SEQ ID NO: 7 - DNA, 309 bp, Artificial Sequence
  PB 5′ITR
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 8 - DNA, 238 bp, Artificial Sequence
  PB 3′ITR
taaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg